(19) United States
(12) Patent Application Publication
(10) Pub. No.: US 2021/0161189 A1
(43) Pub. Date: Jun. 3, 2021

(54) MONOESTER SUGAR DERIVATIVES AS FLAVOR MODIFIERS

(71) Applicant: Firmenich SA, Satigny (CH)

(72) Inventors: Eric FREROT, Geneva (CH); Maxime DELATTRE, Geneva (CH); Angela DI PIETRO, Geneva (CH); Maude GAILLARD, Geneva (CH); Isabelle CAYEUX, Geneva (CH); Philipp ERNI, Geneva (CH); Valeria LARCINESE-HAFNER, Geneva (CH)

(73) Assignee: Firmenich SA, Satigny (CH)

(21) Appl. No.: 17/045,072

(22) PCT Filed: Jul. 31, 2019
(86) PCT No.: PCT/EP2019/070664

§ 371 Date: Oct. 2, 2020

Related U.S. Application Data
(60) Provisional application No. 62/714,047, filed on Aug. 2, 2018.

(30) Foreign Application Priority Data
Aug. 22, 2018 (EP) 18190305.5

(51) Int. Cl. A23L 27/30; C07H 13/06; C12P 19/44

(52) U.S. Cl. CPC A23L 27/33; C12Y 301/01003; C12P 19/44; C07H 13/06

(57) ABSTRACT

Compositions containing monoester derivatives of the primary alcohol residue of a sugar are generally disclosed herein, as well as their use as sweetness enhancers, bitterness maskers, sourness maskers, or astringency reducers, to improve the taste profile of a flavored article.

### CROSS-REFERENCE TO RELATED APPLICATIONS

The present application claims priority to U.S. Provisional Application No. 62/714,047, filed Aug. 2, 2018, and European Patent Application No. 18190305.5, filed Aug. 22, 2018, both of which are hereby incorporated by reference as though set forth herein in their entireties.

### TECHNICAL FIELD

Compositions containing monoester derivatives of the primary alcohol residue of a sugar are generally disclosed herein, as well as their use as sweetness enhancers, bitterness maskers, sourness maskers, or astringency reducers, to improve the taste profile of a flavored article.

### BACKGROUND

Flavor modifiers are substances added to supplement, enhance, or modify the original flavor of a flavored article. Flavor is defined as the combined perception of taste, smell or aroma and chemical feeling factors. The perception of flavor is a result of the chemical stimulation of receptors in both the oral and nasal cavities. The basic tastes are sweet, sour, salty and bitter. Umami, described as another basic taste, enhances the taste effect of other ingredients and components of the flavor profile. These basic tastes, including Umami and certain trigeminal effects are perceived in the buccal cavity. Aroma may be the smell emanating from food before it is consumed or the flavor perceived while chewing and swallowing a product.

Flavor or aroma modifiers may be added to foods (including beverages), personal or household care products, pharmaceutical preparations, or other compositions to increase acceptance of products by enhancing desirable flavors or aromas or by masking or eliminating undesirable attributes. Flavor modifiers may be used to alter the taste or aroma of flavored articles, such as ingestible foods, nutraceuticals and pharmaceuticals, as well as oral and personal care products (e.g., mouthwash, toothpaste, cosmetics, perfumes and the like), or products that may be found in and around homes, businesses, and the like.

Certain lipidated esters of sugars, such as lipidated glucose esters, can be used as surfactants and emulsifiers. In some cases, such emulsifiers can be used in food products, for example, where it may be desirable to enhance the blending of oily substances into aqueous media during the production process. Certain such uses of lipidated glucose esters are described in European Patent Publication No. 0 428 157. But these uses generally demand higher concentrations of the compounds than what is provided by the present disclosure. The use of certain such lipidated glucose esters as flavor modifiers at low concentration was not appreciated therein.

Developing safe and effective compounds to modify and enhance the flavor profile of flavored articles continues to pose certain challenges. It is desirable to discover natural and safe compounds that can serve that role.

### SUMMARY

It was recently discovered that certain esters formed at the primary alcohol of a sugar compound, when used at sufficiently low concentrations, function effectively as taste modifiers and can improve the flavor profile of flavored food and beverage items, particularly items that use artificial low-calorie sweeteners. The disclosure provides certain such compounds, flavored articles containing such compounds, and uses of such compounds to improve the flavor profile of food and beverage items.

In a first aspect, the disclosure provides flavor-enhancing compounds, which are monoester derivatives of a primary alcohol residue of a sugar compound. In some embodiments, the sugar compound is glucose, where the ester forms at the primary alcohol attached to the carbon at the 6-position of the glucose compound. Such derivatives are typically formed from organic acids, such as fatty acids, and any suitable esters thereof.

In a second aspect, the disclosure provides a flavored article comprising one or more flavor-enhancing compounds of the first aspect. In some embodiments, the flavor-enhancing compounds are present in the flavored article at a concentration of no more than 950 ppm, based on the total weight of the flavored article.

In a third aspect, the disclosure provides methods for improving a flavor profile of a flavored article, the method comprising: providing a flavored article, and introducing one or more flavor-enhancing compounds of the first aspect to the flavored article. In some embodiments, the one or more flavor-enhancing compounds are introduced at a concentration of no more than 950 ppm, based on the total weight of the flavored article.

In a fourth aspect, the disclosure provides methods for improving a flavor profile of a flavored article, the method comprising: providing a mixture comprising one or more ingredients for making a flavored article, and introducing one or more flavor-enhancing compounds of the first aspect to the mixture to form a flavor-enhanced mixture; and using the flavor-enhanced mixture to form a flavored article. In some embodiments, the one or more flavor-enhancing compounds are introduced at an effective amount, such as an olfactory or gustatory effective amount, such as at a concentration of no more than 950 nm, based on the total weight of the flavored article.

In a fifth aspect, the disclosure provides methods for improving a flavor perception of a flavored article in a subject (such as a subject in need thereof), the method comprising: administering a flavored article to a subject, wherein the flavored article comprises one or more flavor-enhancing compounds of the first aspect. In some embodiments, the one or more flavor-enhancing compounds are present in the flavored article at an effective amount, such as an olfactory or gustatory effective amount, such as at a concentration of no more than 950 ppm, based on the total weight of the flavored article.

In a sixth aspect, the disclosure provides methods for reducing or masking an off-note of an artificial sweetener, the method comprising: providing a composition comprising an artificial sweetener, and introducing one or more flavor-enhancing compounds of the first aspect to the composition. In some embodiments, the one or more flavor-enhancing compounds are introduced at an effective amount, such as an olfactory or gustatory effective amount, such as at a concentration of no more than 950 ppm, based on the total weight of the composition.

In a seventh aspect, the disclosure provides methods for reducing or masking an off-note perception of an artificial sweetener in a subject (such as a subject in need thereof), the method comprising: administering a composition to a subject, wherein the composition comprises one or more flavor-enhancing compounds of the first aspect and an artificial sweetener. In some embodiments, the one or more flavor-enhancing compounds are present in the composition at an effective amount, such as an olfactory or gustatory effective amount, such as at a concentration of no more than 950 ppm, based on the total weight of the composition.

In each of the foregoing aspects, the flavor-enhancing compounds of the first aspect can be provided in any suitable manner For example, in some embodiments, they are formulated as part of a composition, where the composition comprises one or more flavor-enhancing compounds of the first aspect. In some such embodiments, the composition further comprises a carrier, such as a liquid carrier (e.g., water) or a solid carrier (e.g., starch, dextrose, cellulose, or the like). In some embodiments, such compositions include one or more sweeteners, such as sucrose, high fructose corn syrup, steviol glycosides, rebausiosides, luo han guo extract, mogrosides, and the like, or any mixtures thereof.

In certain of the foregoing aspects, the methods include improving a flavor profile or improving a perception of a flavor profile. In some embodiments, such methods comprise one or more of: reducing or masking bitterness or the perception of bitterness; reducing or masking sourness or the perception of sourness; a reducing or masking astringency or the percention of astringency; enhancing, increasing, or improving sweetness or the perception of sweetness; or any combination thereof.

The flavor-enhancing compounds can be generated in any suitable way. In some embodiments, the flavor-enhancing compounds are generated via the enzymatic esterification of a sugar using an acid donor molecule.

The flavor-enhancing compounds contain, among other features, a sugar moiety and a tail moiety contributed by an acid (or ester thereof). The sugar moiety can be derived from any suitable sugar. Non-limiting examples of such sugars are fructose, sorbose, tagatose, psicose, allose, altrose, glucose, mannose, gulose, idose, galactose, and talose. Non-limiting examples of the tail moiety contributed by an acid are acetic acid, hexanoic acid, octanoic acid, decanoic acid, oleic acid, palmitic acid, hexadecanoic acid, lauric acid, myristic acid, the fatty acids of butter oil, the fatty acids of olive oil, phenylpropanoic acid, cinnamic acid, caffeic acid, gallic acid, ferulic acid, 9-decenoic acid, 10-undecenoic acid, stearic acid, 9-dodecenoic acid, and dodecanoic acid. In some further embodiments, the flavor-enhancing compounds are selected from the group consisting of: glucose-6-O-acetate, glucose-6-O-hexanoate, glucose-6-octanoate, glucose-6-O-decanoate, glucose-6-O-hexadecanoate, glucose-6-O-oleate, glucose-6-O-laurate, glucose-6-O-myristate, glucose-6-O-palmitate, glucose-6-O-phenylpropanoate, glucose-6-O-cinnamate, and any mixtures thereof.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows a graph indicating the sensory properties of a dairy test composition (milk sweetened with 3% w/v sucrose) containing 100 ppm glucose-6-O-octanoate (71007), compared to a control diary composition.

FIG. 2 shows a graph indicating the sensory properties of a coffer drink test composition (coffee containing milk) containing 200 ppm glucose-6-O-octanoate, compared to a control coffee drink composition.

FIG. 3 shows a graph indicating the sensory properties of a strawberry flavored bottled water test composition (strawberry BWAT containing 7% w/v sucrose) containing 100 ppm glucose-6-O-octanoate, compared to a control strawberry flavored bottled water composition.

FIG. 4 shows a graph indicating the sensory properties of a lemon flavored alcoholic beverage test composition containing 100 ppm glucose-6-O-octanoate, compared to a control lemon flavored alcoholic beverage composition.

FIG. 5 shows a graph indicating the sensory properties of a lemon flavored alcoholic beverage test composition containing 200 ppm glucose-6-O-octanoate, compared to a control lemon flavored alcoholic beverage composition.

FIG. 6 shows a graph indicating the sensory properties of a pomegranate juice test composition containing 100 ppm glucose-6-O-octanoate, compared to a control pomegranate juice composition.

FIG. 7 shows a graph indicating the sensory properties of an energy drink test composition containing 100 ppm glucose-6-O-octanoate, compared to a control energy drink composition.

FIG. 8 shows a graph indicating the sensory properties of an energy drink test composition containing 200 ppm glucose-6-O-octanoate, compared to a control energy drink composition.

FIG. 9 shows a graph indicating the sensory properties of a raspberry flavored water test composition containing 100 ppm glucose-6-O-octanoate, compared to a control raspberry flavored water composition.

FIG. 10 shows a graph indicating the sensory properties of a raspberry flavored water test composition containing 200 ppm glucose-6-O-octanoate, compared to a control raspberry flavored water composition.

FIG. 11 shows a graph indicating the sensory properties of an orange flavored test composition containing 200 ppm glucose-6-O-octanoate, compared to a control orange flavored composition.

FIG. 12 shows a graph indicating the sensory properties of 100 ppm glucose-6-O-octanoate in the indicated model systems: (a) SG95 (a composition comprising 95% pure steviol glycoside) 0.02%, (b) SG95 0.03%, citric acid 0.15%, (c) sucrose 4% and sucrose 5%/citric acid 0.15%, (d) grapefruit beverage containing inverted sugar 7%, citric acid 0.15%, grapefruit flavor 0.01%, (e) semi-skimmed milk, with and without 4% sucrose.

FIG. 13 shows a graph indicating the sensory properties of 100 ppm glucose-6-O-acetate in the indicated model systems: sucrose 4%, SG95 (a composition comprising 95% pure steviol glycoside) 0.02%; bitter cocktail with paracetamol 0.09%; quinine 0.0007%; sucrose 5%/citric acid 0.15%.

FIG. 14 shows a graph indicating the sensory properties of 100 ppm glucose-6-O-acetate in the indicated model systems: sucrose 4%; SG95 (a composition comprising 95% pure steviol glycoside) 0.02%; bitter cocktail with paracetamol 0.09%, quinine 0.0007%; sucrose 5%/citric acid 0.15%; SG95 0.03%; citric acid 0.15%; grapefruit beverage containing inverted sugar 7%; citric acid 0.15%, and grapefruit flavor 0.01%.

FIG. 15 shows a graph indicating the sensory properties of 100 ppm glucose-6-O-decanoate in the indicated model systems: sucrose 4%, SG95 (a composition comprising 95% pure steviol glycoside) 0.02%; bitter cocktail with paracetamol 0.09%, quinine 0.0007%; sucrose 5%/citric acid 0.15%; SG95 0.03%; citric acid 0.15%; grapefruit beverage containing inverted sugar 7%; citric acid 0.15%, and grapefruit flavor 0.01%.

FIG. 16 shows a graph indicating the sensory properties of 100 ppm glucose-6-O-hexadecanoate in the indicated model systems: sucrose 4%, SG95 (a composition comprising 95% pure steviol glycoside) 0.02%; bitter cocktail with paracetamol 0.09%, quinine 0.0007%; sucrose 5%/citric acid 0.15%; SG95 0.03%; citric acid 0.15%; grapefruit beverage containing inverted sugar 7%; citric acid 0.15%, and grapefruit flavor 0.01%.

FIG. 17 shows a graph indicating the sensory properties of 100 ppm glucose-6-O-oleate in the indicated model systems: sucrose 4%, SG95 (a composition comprising 95% pure steviol glycoside) 0.02%; bitter cocktail with paracetamol 0.09%, quinine 0.0007%; sucrose 5%/citric acid 0.15%; SG95 0.03%; citric acid 0.15%; grapefruit beverage containing inverted sugar 7%; citric acid 0.15%, and grapefruit flavor 0.01%.

FIG. 18 shows a graph indicating the sensory properties of 100 ppm glucose-6-O-oleyl derivative in the indicated model systems: sucrose 4%, SG95 (a composition comprising 95% pure steviol glycoside) 0.02%; bitter cocktail with paracetamol 0.09%, quinine 0.0007%; sucrose 5%/citric acid 0.15%; SG95 0.03%; citric acid 0.15%; grapefruit beverage containing inverted sugar 7%; citric acid 0.15%, and grapefruit flavor 0.01%.

### DETAILED DESCRIPTION

In the following description, reference is made to specific embodiments which may be practiced, which is shown by way of illustration. These embodiments are described in detail to enable those skilled in the art to practice the various inventions described herein, and it is to be understood that other aspects and embodiments may be used and that logical changes may be made without departing from the scope of the aspects and embodiments presented herein. The following description is, therefore, not to be taken in a limited sense, and the scope of the various aspects presented herein is defined by the appended claims.

The Abstract is provided to allow the reader to quickly ascertain the nature and gist of the technical disclosure. The Abstract is submitted with the understanding that it will not be used to limit the scope or meaning of the claims.

It has long been a goal to improve the quality of food products and to provide new and different flavor and aroma sensations to such products. Commercial production of foods expected to have a relatively long shelf-life often requires the use of processing conditions, storage conditions or addition of ingredients that may produce undesirable off-tastes in the food compositions. Typical solutions to taste problems are ineffective often due to the high cost of ingredients and manufacturing. The use of flavor modifiers could eliminate or substantially reduce the undesirable off-tastes in food compositions as well as improve the overall taste perception of the food or provide new and novel taste experiences.

Certain flavored articles may contain highly bitter, astringent, or sour ingredients (e.g., caffeine, quinine, citric acid, malic acid, tartaric acid, KCl, and the like). In such flavored articles, in order to increase a consumer's liking of the flavored article, it may be desirable to mask the bitterness and modulate the astringency or sourness of the highly bitter, astringent, or sour ingredients. Furthermore, it may also be desirable to enhance the sweetness of a flavored article that contains highly bitter, astringent, or sour ingredients.

### Flavor-Enhancing Compounds

In a first aspect, the disclosure provides flavor-enhancing compounds, which are monoester derivatives of a primary alcohol residue of a sugar compound. In some embodiments, the sugar compound is glucose, where the ester forms at the primary alcohol attached to the carbon at the 6-position of the glucose compound. Such derivatives are typically formed from organic acids, such as fatty acids, and any suitable esters thereof.

The flavor-enhancing compounds disclosed herein may be generated in any suitable manner Thus, in certain aspects, this disclosure provides methods of generating such compounds by a process that comprises enzymatic esterification of a sugar using an acid donor molecule. In some embodiments thereof, the enzyme is a lipase. In one further such embodiment, the lipase is obtained from Candida antartica.

The sugar moiety of the ester can be that of any suitable sugar having a primary alcohol in its free (non-esterified) form. For example, in some embodiments, the sugar moiety is a moiety of a sugar selected from the group consisting of: fructose, sorbose, tagatose, psicose, allose, altrose, glucose, mannose, gulose, idose, galactose, and talose. In some embodiments, the sugar moiety is a glucose moiety.

The acid moiety of the ester can be that of any suitable organic acid, such as an organic carboxylic acid, including the moieties of any natural fatty acid. In some embodiments, the acid moiety is a moiety of an organic carboxylic acid selected from the group consisting of: acetic acid, hexanoic acid, octanoic acid, decanoic acid, oleic acid, palmitic acid, hexadecanoic acid, lauric acid, myristic acid, fatty acids of butter oil, fatty acids of olive oil, phenylpropanoic acid, cinnamic acid, caffeic acid, gallic acid, and ferulic acid. In some other embodiments, the acid moiety is a moiety of an organic carboxylic acid selected from the group consisting of: 9-decenoic acid, 10-undecenoic acid, 9-dodecenoic acid, and dodecanoic acid. In some embodiments, the acid moiety is a moiety of octanoic acid. In some other embodiments, the acid moiety is a moiety of an organic carboxylic acid selected from the fatty acids of fish or hill oil.

In some embodiments of the process of making the compounds, it is desirable to limit reaction to the primary alcohol group of the sugar. Thus, in some such embodiments, the reaction is carried out in the presence of an enzyme. In some such embodiments, the enzyme substantially limits esterification of ant hydroxyl groups on the sugar except for those having primary alcohol functionality. Thus, for example, where the sugar is selected from the group consisting of: allose, altrose, glucose, mannose, gulose, idose, galactose, and talose, the esterification using such enzymatic methods would result in esterification with the donor acyl group attaching at the 6-OH position of the sugar compound.

Where, for example, the sugar is selected from the group consisting of fructose, sorbose, tagatose, psicose, esterification using such enzymatic methods would result in esterification by the donor acyl group at the 1-OH or the 6-OH residue. Accordingly, in some embodiments, the resulting composition is a monoester derivative where the donor acyl group is covalently attached to the 1-OH position of the sugar. Alternatively, in some other such embodiments, the resulting composition may be a monoester derivative where the donor acyl group is covalently attached to the 6-OH position of the sugar. Alternatively, in some embodiments, the resulting composition may be a diester derivative where a donor acyl group is covalently attached to both the 1-OH and the 6-OH positions of the sugar. Thus, in some embodiments, the resulting composition is a mixture of any of the monoester and diester derivatives.

The esters disclosed herein can also be made in other ways. For example, in some other embodiments, the flavor-enhancing compounds are generated by incubating a sugar with tert-butanol and an acid donor in the presence of a lipase. In some such embodiments, the sugar, tert-butanol, the acid donor, and the lipase mixture is incubated at no more than 50° C. for up to 4 days. After incubation, the sugar, tert-butanol, acid donor, and lipase mixture are filtered, rinsed with ethanol, and concentrated to obtain a concentrated reaction product. In some aspects, the concentrated reaction product is dissolved in ethyl acetate and applied to a silica column. In some further embodiments, any unreacted acid donor in the concentrated reaction product is removed by washing the silica column with ethyl acetate. In some embodiments, the flavor-enhancing compounds are purified from the concentrated reaction product by applying a 1:1 ratio of ethyl acetate/ethanol to the silica column, thereby eluting the at least one molecule comprising a monoester derivative of a primary alcohol residue of a sugar off the column. The eluted flavor-enhancing compounds are then concentrated, dissolved in water, and stored. The solution of the flavor-enhancing compounds is, in some further embodiments, freeze dried.

In some embodiments, for example, glucose-6-O-acetate is generated by incubating glucose with tert-butanol and acetic acid in the presence of a lipase. In some further embodiments thereof, the lipase is obtained from Candida antartica. In some further embodiments, the glucose, tert-butanol, acetic acid, and lipase mixture is incubated at a temperature that does not exceed 50° C., for up to 4 days. After incubation, the glucose, tert-butanol, acetic acid, and lipase mixture are filtered, rinsed with a solvent, such as, for example, an alcohol (e.g. ethanol), and concentrated to obtain a concentrated reaction product. In some embodiments, the concentrated reaction product is dissolved in ethyl acetate and applied to a silica column. In some embodiments, any unreacted acetic acid in the concentrated reaction product is removed by washing the silica column with ethyl acetate. In some embodiments, the glucose-6-O-acetate is purified from the concentrated reaction product by applying a 1:1 ratio of ethyl acetate/ethanol to the silica column, thereby eluting the glucose-6-O-acetate off the column. The eluted glucose-6-O-acetate is then concentrated, dissolved in water and stored. In some embodiments, the solution of glucose-6-O-acetate is also freeze dried.

In another embodiment, glucose-6-O-hexanoate is generated by incubating glucose with tert-butanol and hexanoic acid in the presence of a lipase. In some embodiments thereof, the lipase is obtained from Candida antartica. In some embodiments, the glucose, tert-butanol, hexanoic acid and lipase mixture are incubated at no more than 50° C. for up to 4 days. After incubation, the glucose, tert-butanol, hexanoic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted hexanoic acid in the concentrated reaction product is removed by washing the silica column with ethyl acetate. In some embodiments, glucose-6-O-hexanoate is purified from the concentrated reaction product by applying a 1:1 ratio of ethyl acetate/ethanol to the silica column, thereby eluting the glucose-6-O-hexanoate off the column. The eluted glucose-6-O-hexanoate is then concentrated, dissolved in water and stored. In some embodiments, the solution of glucose-6-O-hexanoate is also freeze dried.

In another embodiment, glucose-6-O-octanoate is generated by incubating glucose with tert-butanol and octanoic acid in the presence of a lipase. In some embodiments thereof, he lipase is obtained from Candida antartica. In some embodiments, the glucose, tert-butanol, octanoic acid and lipase mixture may be incubated at no more than 50° C. for up to 4 days. After incubation, the glucose, tert-butanol, octanoic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted octanoic acid in the concentrated reaction product is removed by washing the silica column with ethyl acetate. In some embodiments, glucose-6-O-octanoate is purified from the concentrated reaction product by applying a 1:1 ratio of ethyl acetate/ethanol to the silica column, thereby eluting the glucose-6-O-octanoate off the column. The eluted glucose-6-O-octanoate is then concentrated, dissolved in water and stored. In some embodiments, the solution of glucose-6-O-octanoate is freeze dried.

In another embodiments, glucose-6-O-decanoate is generated by incubating glucose with tert-butanol and decanoic acid in the presence of a lipase. In some embodiments thereof, the lipase is obtained from Candida antartica. In some embodiments, the glucose, tert-butanol, decanoic acid and lipase mixture are incubated at no more than 50° C. for up to 4 days. After incubation, the glucose, tert-butanol, decanoic acid and lipase mixture is filtered, rinsed with ethanol and concentrated to obtain a concentrated reaction product. In some embodiments, the concentrated reaction product is dissolved in ethyl acetate and applied to a silica column. In some embodiments, any unreacted decanoic acid in the concentrated reaction product is removed by washing the silica column with ethyl acetate. In some embodiments, glucose-6-O-decanoate is purified from the concentrated reaction product by applying a 1:1 ratio of ethyl acetate/ethanol to the silica column, thereby eluting the glucose-6-O-decanoate off the column. The eluted glucose-6-O-decanoate may then be concentrated, dissolved in water and stored. In some embodiments, the solution of glucose-6-O-decanoate is freeze dried.

In another embodiment, glucose-6-O-hexadecanoate is generated by incubating glucose with tert-butanol and hexadecanoic acid in the presence of a lipase. In some embodiments, the lipase is obtained from Candida antartica. In some embodiments, the glucose, tert-butanol, hexadecanoic acid and lipase mixture is incubated at no more than 50° C. for up to 4 days. After incubation, the glucose, tert-butanol, hexadecanoic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted hexadecanoic acid in the concentrated reaction product is removed by washing the silica column with ethyl acetate. In some embodiments, glucose-6-O-hexadecanoate is purified from the concentrated reaction product by applying a 1:1 ratio of ethyl acetate/ethanol to the silica column, thereby eluting the glucose-6-O-hexadecanoate off the column. The eluted glucose-6-O-hexadecanoate is then concentrated, dissolved in water and stored. In some embodiments, the solution of glucose-6-O-hexadecanoate is freeze dried.

In another embodiment, glucose-6-O-oleate is generated by incubating glucose with tert-butanol and oleic acid in the presence of a lipase. In some embodiments thereof, the lipase is obtained from Candida antartica. In some embodiments, the glucose, tert-butanol, oleic acid and lipase mixture are incubated at no more than 50° C. for up to 4 days. After incubation, the glucose, tert-butanol, oleic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted oleic acid in the concentrated reaction product is removed by washing the silica column with ethyl acetate. In some embodiments, glucose-6-O-oleate is purified from the concentrated reaction product by applying a 1:1 ratio of ethyl acetate/ethanol to the silica column, thereby eluting the glucose-6-O-oleate off the column. The eluted glucose-6-O-oleate is then concentrated, dissolved in water and stored. In some embodiments, the solution of glucose-6-O-oleate may also be freeze dried.

In another embodiment, glucose-6-O-laurate is generated by incubating glucose with tert-butanol and lauric acid in the presence of a lipase. In some embodiments, the lipase is obtained from Candida antartica. In some embodiments, the glucose, tert-butanol, lauric acid and lipase mixture are incubated at no more than 50° C. for up to 4 days. After incubation, the glucose, tert-butanol, lauric acid and lipase mixture is filtered, rinsed with ethanol and concentrated to obtain a concentrated reaction product. In some embodiments, the concentrated reaction product is dissolved in ethyl acetate and applied to a silica column. In some embodiments, any unreacted lauric acid in the concentrated reaction product is removed by washing the silica column with ethyl acetate. In some embodiments, glucose-6-O-laurate is purified from the concentrated reaction product by applying a 1:1 ratio of ethyl acetate/ethanol to the silica column, thereby eluting the glucose-6-O-laurate off the column. The eluted glucose-6-O-laurate is then concentrated, dissolved in water and stored. In some embodiments, the solution of glucose-6-O-laurate is freeze dried.

In another embodiment, glucose-6-O-myristate is generated by incubating glucose with tert-butanol and myristic acid in the presence of a lipase. In some embodiments thereof, the lipase is obtained from Candida antartica. In some embodiments, the glucose, tert-butanol, myristic acid and lipase mixture is incubated at no more than 50° C. for up to 4 days. After incubation, the glucose, tert-butanol, myristic acid and lipase mixture may be filtered, rinsed with ethanol and concentrated to obtain a concentrated reaction product. In some embodiments, the concentrated reaction product is dissolved in ethyl acetate and applied to a silica column. In some embodiments, any unreacted myristic acid in the concentrated reaction product is removed by washing the silica column with ethyl acetate. In some embodiments, glucose-6-O-myristate is purified from the concentrated reaction product by applying a 1:1 ratio of ethyl acetate/ethanol to the silica column, thereby eluting the glucose-6-O-myristate off the column. The eluted glucose-6-O-myristate is then concentrated, dissolved in water and stored. In some embodiments, the solution of glucose-6-O-myristate may also be freeze dried.

In another embodiment, glucose-6-O-palmitate is generated by incubating glucose with tert-butanol and palmitic acid in the presence of a lipase. In some embodiments thereof, the lipase is obtained from Candida antartica. In some embodiments, the glucose, tert-butanol, palmitic acid and lipase mixture are incubated at no more than 50° C. for up to 4 days. After incubation, the glucose, tert-butanol, palmitic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted palmitic acid in the concentrated reaction product is removed by washing the silica column with ethyl acetate. In some embodiments, glucose-6-O-palmitate is purified from the concentrated reaction product by applying a 1:1 ratio of ethyl acetate/ethanol to the silica column, thereby eluting the glucose-6-O-palmitate off the column. The eluted glucose-6-O-palmitate is then concentrated, dissolved in water and stored. In tome embodiments, the solution of glucose-6-O-palmitate is freeze dried.

In another embodiment, glucose-6-O-phenylpropanoate is generated by incubating glucose with tert-butanol and phenylpropanoic acid in the presence of a lipase. In some embodiments thereof, the lipase is obtained from Candida antartica. In some embodiments, the glucose, tert-butanol, phenylpropanoic acid and lipase mixture are incubated at no more than 50° C. for up to 4 days. After incubation, the glucose, tert-butanol, phenylpropanoic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted phenylpropanoic acid in the concentrated reaction product is removed by washing the silica column with ethyl acetate. In some embodiments, glucose-6-O-phenylpropanoate is purified from the concentrated reaction product by applying a 1:1 ratio of ethyl acetate/ethanol to the silica column, thereby eluting the glucose-6-O-phenylpropanoate off the column. The eluted glucose-6-O-phenylpropanoate is then concentrated, dissolved in water and stored. In some embodiments, the solution of glucose-6-O-phenylpropanoate is freeze dried.

In another embodiment, glucose-6-O-cinnamate is generated by incubating glucose with tert-butanol and cinnamic acid in the presence of a lipase. In some embodiments thereof, the lipase is obtained from Candida antartica. In some embodiments, the glucose, tert-butanol, cinnamic acid and lipase mixture are incubated at no more than 50° C. for up to 4 days. After incubation, the glucose, tert-butanol, cinnamic acid and lipase mixture are filtered, rinsed with ethanol and concentrated to obtain a concentrated reaction product. In some embodiments, the concentrated reaction product is dissolved in ethyl acetate and applied to a silica column. In some embodiments, any unreacted cinnamic acid in the concentrated reaction product is removed by washing the silica column with ethyl acetate. In some embodiments, glucose-6-O-cinnamate is purified from the concentrated reaction product by applying a 1:1 ratio of ethyl acetate/ethanol to the silica column, thereby eluting the glucose-6-O-phenylpropanoate off the column. The eluted glucose-6-O-cinnamate is then concentrated, dissolved in water and stored. In some embodiments, the solution of glucose-6-O-cinnamate is freeze dried.

In other embodiments, other monoester derivatives of the primary alcohol residue of glucose are generated using triglycerides as acid donors, such as, the triglycerides found in butter oil, or olive oil, for example. Alternatively, in some further embodiments, other monoester derivatives of the primary alcohol residue of glucose may be generated using butter oil or olive oil as the acid donor.

In other embodiments, the methods outlined above are used to generate monoester derivatives of a primary alcohol residues other sugars, such as, for example, fructose, sorbose, tagatose, psicose, allose, altrose, mannose, gulose, idose, galactose, and talose.

In some embodiments, the at least one molecule comprising a monoester derivative of a primary alcohol residue of a sugar is selected from the group consisting of: glucose-6-O-acetate, glucose-6-O-hexanoate, glucose-6-octanoate, glucose-6-O-decanoate, glucose-6-O-hexadecanoate, glucose-6-O-oleate, glucose-6-laurate, glucose-6-myristate, glucose-6-palmitate, glucose-6-phenylpropanoate, glucose-6-cinnamate, and mixtures thereof.

In some embodiments, the at least one molecule comprising a monoester derivative of a primary alcohol residue of a sugar is generated according to the methods disclosed in Handayani et al., IOSR Journal of Applied Chemistry, vol. 1(6), pp. 45-50 (2012).

In some alternate embodiments, the at least one molecule comprising a monoester derivative of a primary alcohol residue of a sugar is generated by treating a sucrose ester with invertase, thereby generating a mixture of monoesters of fructose and glucose. Sucrose is a disaccharide formed from condensation of glucose and fructose to produce α-D-glucopyranosyl-(1→2)-β-D-fructofuranoside. Sucrose has 8 hydroxyl groups which can be reacted with fatty acid esters to produce sucrose esters. Among the 8 hydroxyl groups on sucrose, three (C6, C1′, and C6′) are primary while the others (C2, C3, C4, C3′, and C4′) are secondary. The three primary hydroxyl groups are more reactive due to lower steric hindrance, so they react with fatty acids first, resulting in a sucrose mono-, di-, or triester. Typical saturated fatty acids that are used to produce sucrose esters are lauric acid, myristic acid, palmitic acid, stearic acid and behenic acid, and typical unsaturated fatty acids are oleic acid and erusic acid.

In one embodiment, the at least one molecule comprising a monoester derivative of a primary alcohol residue of a sugar has the following structure:

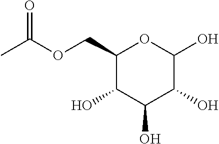

In one embodiment, the at least one molecule comprising a monoester derivative of a primary alcohol residue of a sugar has the following structure:

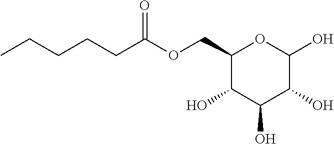 [SMILES: CCCCCC(=O)OC[C@H]1OC(O)[C@H](O)[C@@H](O)[C@@H]1O]

In one embodiment, at least one molecule comprising a monoester derivative of a primary alcohol residue of a sugar has the following structure:

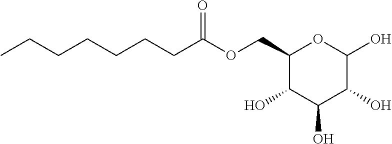 [SMILES: CCCCCCCC(=O)OC[C@H]1OC(O)[C@H](O)[C@@H](O)[C@@H]1O]

In one embodiment, the at least one molecule comprising a monoester derivative of a primary alcohol residue of a sugar has the following structure:

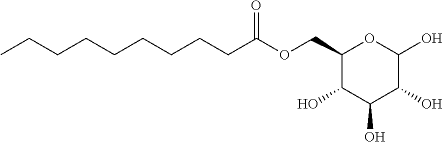 [SMILES: CCCCCCCCCC(=O)OC[C@H]1OC(O)[C@H](O)[C@@H](O)[C@@H]1O]

In one embodiment, the at least one molecule comprising a monoester derivative of a primary alcohol residue of a sugar has the following structure:

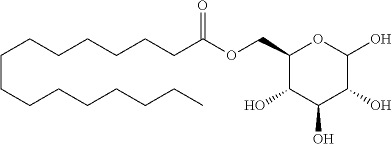 [SMILES: CCCCCCCCCCCCCCCC(=O)OC[C@H]1OC(O)[C@H](O)[C@@H](O)[C@@H]1O]

In one embodiment, the at least one molecule comprising a monoester derivative of a primary alcohol residue of a sugar has the following structure:

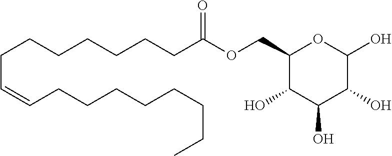 [SMILES: CCCCCCCC/C=C\CCCCCCCC(=O)OC[C@H]1OC(O)[C@H](O)[C@@H](O)[C@@H]1O]

In one embodiment, the at least one molecule comprising a monoester derivative of a primary alcohol residue of a sugar has the following structure:

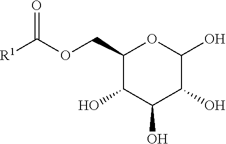

In one embodiment, the at least one molecule comprising a monoester derivative of a primary alcohol residue of a sugar has the following structure:

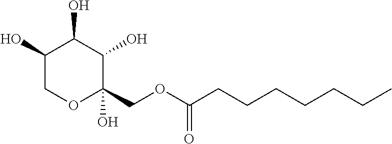 [SMILES: CCCCCCCC(=O)OC[C@@]1(O)OC[C@@H](O)[C@@H](O)[C@@H]1O]

In one embodiment, the at least one molecule comprising a monoester derivative of a primary alcohol residue of a sugar has the following structure:

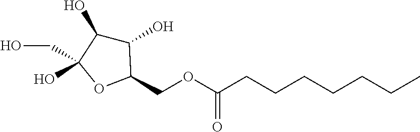 [SMILES: CCCCCCCC(=O)OC[C@H]1O[C@](O)(CO)[C@@H](O)[C@@H]1O]

In one embodiment, the at least one molecule comprising a monoester derivative of a primary alcohol residue of a sugar has the following structure:

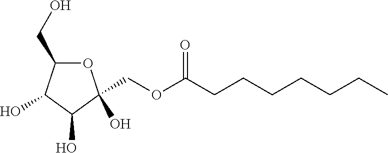 [SMILES: CCCCCCCC(=O)OC[C@@]1(O)O[C@H](CO)[C@@H](O)[C@@H]1O]

In one embodiment, the at least one molecule comprising a monoester derivative of a primary alcohol residue of a sugar has the following structure:

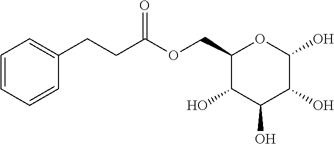 [SMILES: O=C(CCc1ccccc1)OC[C@H]1O[C@H](O)[C@H](O)[C@@H](O)[C@@H]1O]

In one embodiment, the at least one molecule comprising a monoester derivative of a primary alcohol residue of a sugar has the following structure:

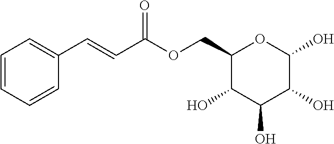 [SMILES: O=C(/C=C/c1ccccc1)OC[C@H]1O[C@H](O)[C@H](O)[C@@H](O)[C@@H]1O]

Accordingly, some embodiments, the disclosure provides a method, wherein the method provides at least one benefit selected from the group consisting of: a reduction or masking of the bitterness of a flavored article, a reduction or masking of the sourness of a flavored article, a reduction or masking of the astringency of a flavored article, an enhancement, increase, or improvement of the sweetness of a flavored article, an improvement of a taste profile of a flavored article, or any combination of benefit thereof, wherein the method comprises the step of adding an olfactory or gustatory effective amount of at least one molecule comprising a monoester derivative of a primary alcohol residue of a sugar to the flavored article.

An some alternate embodiments, the disclosure provides a method, wherein the method reduces or masks the off-notes of an artificial sweetener, wherein the method comprises the step of adding an olfactory or gustatory effective amount of at least one molecule comprising a monoester derivative of a primary alcohol residue of a sugar to the artificial sweetener.

In some embodiments, the artificial sweetener is incorporated into, or added to a flavored article.

Any suitable amount of the flavor-enhancing compounds can be used in any the various aspects and embodiments provided herein. In some embodiments, the flavor-enhancing compounds are present in a concentration the flavor-enhancing compounds are present in a concentration of no more than 1000 ppm, or no more than 950 ppm, or no more than 900 ppm, or no more than 800 ppm, or no more than 750 ppm, or no more than 700 ppm, or no more than 600 ppm or no more than 500 ppm, based on the total weight of the flavored article. In some embodiments, the flavor-enhancing compounds are present in a concentration from 50 to 1000 ppm, or from 50 to 950 ppm, or from 50 to 900 ppm, or from 50 to 800 ppm, or from 50 to 750 ppm, or from 50 to 700 ppm, or from 50 to 600 ppm, or from 50 to 500 ppm, in the flavored article, based on the total weight of the flavored article. Alternatively, the flavor-enhancing compounds are present in a concentration from 60 to 1000 ppm, or from 60 to 950 ppm, or from 60 to 900 ppm, or from 60 to 800 ppm, or from 60 to 750 ppm, or from 60 to 700 ppm, or from 60 to 600 ppm, or from 60 to 500 ppm, in the flavored article. Alternatively, the flavor-enhancing compounds are present in a concentration from 70 to 1000 ppm, or from 70 to 950 ppm, or from 70 to 900 ppm, or from 70 to 800 ppm, or from 70 to 750 ppm, or from 70 to 700 ppm, or from 70 to 600 ppm, or from 70 to 500 ppm, in the flavored article, based on the total weight of the flavored article. Alternatively, the flavor-enhancing compounds are present in a concentration from 80 to 1000 ppm, or from 80 to 950 ppm, or from 80 to 900 ppm, or from 80 to 800 ppm, or from 80 to 750 ppm, or from 80 to 700 ppm, or from 80 to 600 ppm, or from 80 to 500 ppm, in the flavored article, based on the total weight of the flavored article. Alternatively, the flavor-enhancing compounds are present in a concentration from 90 to 1000 ppm, or from 90 to 950 ppm, or from 90 to 900 ppm, or from 90 to 800 ppm, or from 90 to 750 ppm, or from 90 to 700 ppm, or from 90 to 600 ppm, or from 90 to 500 ppm, in the flavored article, based on the total weight of the flavored article. Alternatively, the flavor-enhancing compounds are present in a concentration from 100 to 1000 ppm, or from 100 to 950 ppm, or from 100 to 900 ppm, or from 100 to 800 ppm, or from 100 to 750 ppm, or from 100 to 700 ppm, or from 100 to 600 ppm, or from 100 to 500 ppm, in the flavored article, based on the total weight of the flavored article. Alternatively, the flavor-enhancing compounds are present in a concentration from 150 to 1000 ppm, or from 150 to 950 ppm, or from 150 to 900 ppm, or from 150 to 800 ppm, or from 150 to 750 ppm, or from 150 to 700 ppm, or from 150 to 600 ppm, or from 150 to 500 ppm, in the flavored article, based on the total weight of the flavored article. Alternatively, the flavor-enhancing compounds are present in a concentration from 200 to 1000 ppm, or from 200 to 950 ppm, or from 200 to 900 ppm, or from 200 to 800 ppm, or from 200 to 750 ppm, or from 200 to 700 ppm, or from 200 to 600 ppm, or from 200 to 500 ppm, in the flavored article, based on the total weight of the flavored article. Alternatively, the flavor-enhancing compounds are present in a concentration from 250 to 1000 ppm, or from 250 to 950 ppm, or from 250 to 900 ppm, or from 250 to 800 ppm, or from 250 to 750 ppm, or from 250 to 700 ppm, or from 250 to 600 ppm, or from 250 to 500 ppm, in the flavored article, based on the total weight of the flavored article. Alternatively, the flavor-enhancing compounds are present in a concentration from 300 to 1000 ppm, or from 300 to 950 ppm, or from 300 to 900 ppm, or from 300 to 800 ppm, or from 300 to 750 ppm, or from 300 to 700 ppm, or from 300 to 600 ppm, or from 300 to 500 ppm, in the flavored article, based on the total weight of the flavored article. Alternatively, the flavor-enhancing compounds are present in a concentration from 350 to 1000 ppm, or from 350 to 950 ppm, or from 350 to 900 ppm, or from 350 to 800 ppm, or from 350 to 750 ppm, or from 350 to 700 ppm, or from 350 to 600 ppm, or from 350 to 500 ppm, in the flavored article, based on the total weight of the flavored article. Alternatively, the flavor-enhancing compounds are present in a concentration from 400 to 1000 ppm, or from 400 to 950 ppm, or from 400 to 900 ppm, or from 400 to 800 ppm, or from 400 to 750 ppm, or from 400 to 700 ppm, or from 400 to 600 ppm, or from 400 to 500 ppm, in the flavored article, based on the total weight of the flavored article. Alternatively, the flavor-enhancing compounds are present in a concentration from 450 to 1000 ppm, or from 450 to 950 ppm, or from 450 to 900 ppm, or from 450 to 800 ppm, or from 450 to 750 ppm, or from 450 to 700 ppm, or from 450 to 600 ppm, or from 450 to 500 ppm, in the flavored article, based on the total weight of the flavored article. Alternatively, the flavor-enhancing compounds are present in a concentration from 500 to 1000 ppm, or from 500 to 950 ppm, or from 500 to 900 ppm, or from 500 to 800 ppm, or from 500 to 750 ppm, or from 500 to 700 ppm, or from 500 to 600 ppm, in the flavored article, based on the total weight of the flavored article. Alternatively, the flavor-enhancing compounds are present in a concentration from 550 to 1000 ppm in the flavored article. Alternatively, the flavor-enhancing compounds are present in a concentration from 600 to 1000 ppm in the flavored article. Alternatively, the flavor-enhancing compounds are present in a concentration from 650 to 1000 ppm in the flavored article. Alternatively, the flavor-enhancing compounds are present in a concentration from 700 to 1000 ppm in the flavored article. Alternatively, the flavor-enhancing compounds are present in a concentration from 750 to 1000 ppm in the flavored article. Alternatively, the flavor-enhancing compounds are present in a concentration from 800 to 1000 ppm in the flavored article. Alternatively, the flavor-enhancing compounds are present in a concentration from 850 to 1000 ppm in the flavored article. Alternatively, the flavor-enhancing compounds are present in a concentration from 900 to 1000 ppm in the flavored article. Alternatively, the flavor-enhancing compounds are present in a concentration from 950 to 1000 ppm in the flavored article.

In some embodiments, the flavor-enhancing compounds are present in a concentration of 50 ppm, or 60 ppm, or 70 ppm, or 80 ppm, or 90 ppm, or 100 ppm, or 150 ppm, or 100 ppm, or 150 ppm, or 200 ppm, or 250 ppm, or 300 ppm, or 350 ppm, or 400 ppm, or 450 ppm, or 500 ppm, or 550 ppm, or 600 ppm, or 650 ppm, or 700 ppm, or 750 ppm, or 800 ppm, or 850 ppm, or 900 ppm, or 950 ppm, or 1000 ppm in the flavored article.

In some other embodiments, the disclosure provides a method, wherein the method provides at least one benefit selected from the group consisting of: a reduction or masking of the perception of bitterness in a subject in need thereof, a reduction or masking of the sourness of a flavored article, a reduction or masking of the perception of astringency in a subject in need thereof, an enhancement, increase, or improvement of the perception of sweetness in a subject in need thereof, an improvement of a taste profile of a flavored article perceived by the subject, or any combination of benefit thereof, wherein the method comprises contacting the subject with an olfactory or gustatory effective amount of at least one molecule comprising a monoester derivative of a primary alcohol residue of a sugar.

In some other embodiments, the disclosure provides a method, wherein the method reduces or masks the perception of off-notes of an artificial sweetener in a subject in need thereof, wherein the method comprises contacting the subject with an olfactory or gustatory effective amount of at least one molecule comprising a monoester derivative of a primary alcohol residue of a sugar.

In some embodiments, the artificial sweetener is incorporated into, or added to a flavored article.

In some embodiments, the olfactory or gustatory effective amount of the at least one molecule comprising a monoester derivative of a primary alcohol residue of a sugar at which the subject is contacted with is from 50 to 1000 ppm.

### Compositions

As shown in the Examples below, the present disclosure demonstrates that it was surprisingly and unexpectedly found that compositions comprising at least one molecule comprising a monoester derivative of a primary alcohol residue of a sugar are useful to provide at least one benefit selected from the group consisting of: a reduction or masking of the bitterness of a flavored article, a reduction or masking of the sourness of a flavored article, a reduction or masking of the astringency of a flavored article, an enhancement, increase, or improvement of the sweetness of a flavored article, an improvement of a taste profile of a flavored article, or any combination of benefit thereof.

Furthermore, it was surprisingly and unexpectedly found that compositions comprising at least one molecule comprising a monoester derivative of a primary alcohol residue of a sugar are useful to provide at least one benefit selected from the group consisting of: a reduction or masking of the perception of bitterness in a subject in need thereof, a reduction or masking of the sourness of a flavored article, a reduction or masking of the perception of astringency in a subject in need thereof, an enhancement, increase, or improvement of the perception of sweetness in a subject in need thereof, an improvement of a taste profile of a flavored article perceived by the subject, or any combination of benefit thereof.

Accordingly, in some embodiments, the disclosure provides a composition comprising the at least one molecule comprising a monoester derivative of a primary alcohol residue of a sugar.

In some such embodiments, the composition comprises the at least one molecule comprising a monoester derivative of a primary alcohol residue of a sugar in a purity of greater than about 60% by weight, e.g., greater than about 70% by weight, greater than about 80% by weight, greater than about 90% by weight, greater than about 98% by weight, or greater than about 99% by weight.

In some aspects, the composition further comprises at least one additional sweetener. The at least one additional sweetener may be an artificial sweetener, or, alternatively, a natural sweetener.

In some embodiments, the at least one additional sweetener is selected from the group consisting of: abiziasaponin, abrusosides, in particular abrusoside A, abrusoside B, abrusoside C, abrusoside D, acesulfame potassium, advantame, albiziasaponin, alitame, aspartame, superaspartame, bayunosides, in particular bayunoside 1, bayunoside 2, brazzein, bryoside, bryonoside, bryonodulcoside, carnosifloside, carrelame, curculin, cyanin, chlorogenic acid, cyclamates and its salts, cyclocaryoside I, dihydroquercetin-3-acetate, dihydroflavenol, dulcoside, gaudichaudioside, glycyrrhizin, glycyrrhetin acid, gypenoside, hematoxylin, isomogrosides, in particular iso-mogroside V, lugduname, magap, mabinlins, micraculin, mogrosides (lo han guo), in particular mogroside IV and mogroside V, monatin and its derivatives, monellin, mukurozioside, naringin dihydrochalcone (NarDHC), neohesperidin dihydrochalcone (NDHC), neotame, osladin, pentadin, periandrin I-V, perillartine, D-phenylalanine, phlomisosides, in particular phlomisoside 1, phlomisoside 2, phlomisoside 3, phlomisoside 4, phloridzin, phyllodulcin, polpodiosides, polypodoside A, pterocaryosides, rebaudiosides, in particular rebaudioside A, rebaudioside B, rebaudioside C, rebaudioside D, rebaudioside F, rebaudioside G, rebaudioside H), rubusosides, saccharin and its salts and derivatives, scandenoside, selligueanin A, siamenosides, in particular siamenoside I, stevia, steviolbioside, stevioside and other steviol glycosides, strogines, in particular strogin 1, strogin 2, strogin 4, suavioside A, suavioside B, suavioside G, suavioside H, suavioside I, suavioside J, sucralose, sucronate, sucrooctate, talin, telosmoside A15, thaumatin, in particular thaumatin I and II, trans-anethol, trans-cinnamaldehyde, trilobatin, D-tryptophane, erythritol, galactitol, hydrogenated starch syrups including maltitol and sorbitol syrups, inositols, isomalt, lactitol, maltitol, mannitol, xylitol, arabinose, dextrin, dextrose, fructose, high fructose corn syrup, fructooligosaccharides, fructooligosaccharide syrups, galactose, galactooligosaccharides, glucose, glucose and (hydrogenated) starch syrups/hydrolysates, isomaltulose, lactose, hydrolysed lactose, maltose, mannose, rhamnose, ribose, sucrose, tagatose, trehalose and xylose.

In some further embodiments, the at least one additional sweetener may be selected from the sweeteners disclosed in PCT Publication No. WO2012/107203.

Alternatively, in some other embodiments, the at least one additional sweetener may be selected from the compounds disclosed in PCT Publication No. WO2011/130705.

Alternatively, in some other embodiments, the at least one additional sweetener may be selected from the compounds disclosed in European Patent No. 0 605 261 B1.

Alternatively, in some other embodiments, the at least one additional sweetener may be a polymethoxyflavone selected from the group consisting of: nobiletin, sinensetin, heptamethoxyflavone, and tangeretin.

Alternatively, in some other embodiments, the at least one additional sweetener may be a polymethoxyflavone disclosed in PCT Publication No. WO2012/107203.

Alternatively, in some other embodiments, the at least one additional sweetener may be a polymethoxyflavone disclosed in PCT Publication No. WO2011/130705.

Alternatively, in some other embodiments, the at least one additional sweetener may be a polymethoxyflavone disclosed in European Patent No. 0 605 261 B1.

In some embodiments, the composition further comprises at least one additional sweetness enhancer, e.g., at least two or at least three. Suitable additional sweetness enhancers are well known in the art. In one aspect, the at least one additional sweetness enhancer may be selected from the group consisting of terpenes (such as sesquiterpenes, diterpenes, and triterpenes), flavonoids, amino acids, proteins, polyols, other known natural sweeteners (such as cinnamaldehydes, selligueains and hematoxylins), secodammarane glycosides, and analogues thereof. Exemplary sweetness enhancers include steviol glycoside such as stevioside, steviolbioside, rebaudioside A, rebaudioside B, rebaudioside C, rebaudioside D, rebaudioside E, rebaudioside F, dulcoside A, rubusoside; hernandulcin; pine rosin diterpenoid; mukurozioside; baiyunosdie; phlomisoside, such as phlomisoside I and phlomisodie II; glycyrrhizic acid; periandrins, such as periandrin I, periandrin II, periandrin III, and periandrin IV; osladin; polypodosides, such as polypodoside A and polypodoside B; mogrosides, such as mogroside IV and mogroside V; abrusoside A and abrusosdie B; cyclocariosdies, such as cyclocarioside A and cyclocarioside B; pterocaryoside A and pterocaryoside B; flavonoids, such as phyllodulcin, phloridzin, neoastilbin, and dihydroquercetin acetate; amino acids, such as glycine and monatin; proteins, such as thaumatins (thaumatin I, thaumatin II, thaumatin iii, and thaumatin IV), monellin, mabinlins (mabinlin I and mabinlin II), brazzein, miraculin, and curculin; polyols such as erythritol; cinnamaldehyde; selligueains, such as selligueain A and selligueain B; hematoxylin; and mixtures thereof. Additional exemplary sweetness enhancers include pine rosin diterpenoids; phloridizin; neoastilbin; dihydroquercetin acetate; glycine; erythritol; cinnamaldehyde; selligueain A; selligueain B; hematoxylin; rebaudioside A; rebaudioside B; rebaudioside C; rebaudioside D; rebaudioside E; dulcoside A; steviolbioside; rubusoside; stevia; stevioside; steviol 13-O-β-D-glycoside; mogroside V; Luo Han Guo; siamenoside; siamenoside I; monatin and salts thereof (monatin SS, RR, RS, SR); curculin; glycyrrhizic acid and its salts; thaumatin I; thaumatin II; thaumatin III; thaumatin IV; monellin; mabinlin I; mabinlin II; brazzein; hernandulcin; phyllodulcin; glycyphyllin; phloridzin; trilobatin; baiyunoside; osladin; polypodoside A; polypodoside B; pterocaryoside A; pterocaryoside B; mukurozioside; mukurozioside lib; phlomisoside I; phlomisoside II; periandrin I; periandrin II; periandrin III; periandrin VI; periandrin V; cyclocarioside A; cyclocarioside B; suavioside A; suavioside B; suavioside G; suavioside H; suavioside I; suavioside J; labdane glycosides; baiyunoside; gaudichaudioside A; mogroside IV; iso-mogroside; bryodulcoside; bryobioside; bryoside; bryonoside; carnosifloside V; carnosifioside VI; scandenoside R6; 11-oxomogroside V; abrusoside A; abrusoside B; abrusoside C; abrusoside D; abrusoside E; gypenoside XX; glycyrrhizin; apioglycyrrhizin; araboglycyrrhizin; pentadin; perillaldehyde; rebaudioside F; steviol; 13-[(2-O-(3-O-α-D-glucopyranosyl)-β-D-glucopyranosyl-3-O-β-D-glucopyranosyl-β-D-glucopyranosyl)oxy]kaur-16-en-18-oic acid β-D-glucopyranosyl ester; 13-[(2-O-β-D-glucopyranosyl-3-O-(4-O-α-D-glucopyranosyl)-β-D-glucopyranosyl-β-D-glucopyranosyl)oxy] kaur-16-en-18-oic acid β-D-glucopyranosyl ester; 13-[(3-O-β-D-glucopyranosyl-β-D-glucopyranosyl)oxy]kaur-16-en-18-oic acid β-D-glucopyranosyl ester; 13-hydroxy-kaur-16-en-18-oic acid β-D-glucopyranosyl ester; 13-methyl-16-oxo-17-norkauran-18-oic acid β-D-glucopyranosyl ester; 13-[(2-O-β-D-glucopyranosyl-3-O-β-D-giucopyranosyl-β-D-glucopyranosyl)oxy] kaur-15-en-18-oic acid β-D-glucopyranosyl ester; 13-[(2-O-D-glucopyranosyl-3-O-D-glucopyranosyl-β-D-glucopyranosyl)oxy] kaur-15-en-18-oic acid; 13-[(2-O-β-D-glucopyranosyl-3-O-β-D-glucopyranosyl]-β-D-glucopyranosyl)oxyl-17-hydroxy-kaur-15-en-18-oic acid β-D-glucopyranosyl ester; 13-[(2-O-β-D-glucopyranosyl-3-O-D-glucopyranosyl-β-D-glucopyranosyl)oxy]-16-hydroxy kauran-18-oic acid β-D-glucopyranosyl ester; 13-[(2-O-β-D-glucopyranosyl-3-O-β-D-glucopyranosyl-β-D-glucopyranosyl)oxy]-16-hydroxy kauran-18-oic acid; isosteviol; mogroside IA; mogroside IE; mogroside 11-A; mogroside 11-E; mogroside III; mogroside V; isomogroside V; 11-Oxomogroside; mogrol; 11-oxomogrol; 11-oxomogroside IA; 1-[13-hydroxykaur-16-en-18-oate] β-D-glucopyranuronic acid; 13-[(2-O-β-D-glucopyranosyl β-D-glucopyranosyl)oxy]-17-hydroxy-kaur-15-en-18-oic acid β-D-glucopyranosyl ester; 13-[(2-O-β-D-glucopyranosyl-β-D-glucopyranosyl)oxy] kaur-16-en-18-oic acid-(2-O-β-D-glucopyranosyl-β-D-glucopyranosyl)ester (rebaudioside E); 13-[(2-O-α-L-rhamnopyranosyl-3-O-β-D-glucopyranosyl-β-D-glucopyranosyl)oxy] kaur-16-en-18-oic acid-(2-O-β-D-glucopyranosyl-β-D-glucopyranosyl) ester; 13-[(2-O-β-D-glucopyranosyl-3-O-P-D-glucopyranosyl-β-D-glucopyranosyl)oxy]-kaur-16-en-18-oic acid-(2-O-α-L-rhamnopyranosyl-β-D-glucopyranosyl) ester; 13-[(2-O-β-D-glucopyranosyl β-D-glucopyranosyl)oxy]-17-oxo-kaur-15-en-oic acid β-D-glucopyranosl ester; 13-[(2-O-(6-O-β-D-glucopyranosyl)-β-D-glucopyranosyl-β-D-glucopyranosyl)oxy] kaur-16-en-18-oic acid β-D-glucopyranosyl ester; 13-[(2-O-β-D-glucopyranosyl-3-O-β-D-fructofuranosyl-β-D-glucopyranosyl)oxy] kaur-16-en-18-oic acid β-D-glucopyranosyl ester; 13-[(2-O-β-D-glucopyranosyl-β-D-glucopyranosyl)oxy] kaur-16-en-18-oic acid-(6-O-β-D-xylopyranosyl-β-D-glucopyranosyl) ester; 13-[(2-O-β-D-glucopyranosyl-β-D-glucopyranosyl)oxy] kaur-16-en-18-oic acid-(4-O-(2-O-α-D-glucopyranosyl)-α-D-glucopyranosyl-D-glucopyranosyl) ester; 13-[(2-O-β-D-glucopyranosyl-3-O-P-D-glucopyranosyl-β-D-glucopyranosyl)oxy] kaur-16-en-18-oic acid-(2-O-6-deoxy-β-D-glucopyranosyl-β-D-glucopyranosyl) ester; 13-[(2-O-β-D-glucopyranosyl-β-D-glucopyranosyl)oxy] kaur-15-en-18-oic acid β-D-glucopyranosyl ester; 13-[(2-O-β-D-glucopyranosyl-3-O-β-D-xylopyranosyl-P-D-glucopyranosyl)oxy] kaur-16-en-18-oic acid β-D-glucopyranosyl ester; 13-[(2-O-β-D-xylopyranosyl-β-D-glucopyranosyl)oxy] kaur-16-en-18-oic acid β-D-glucopyranosyl ester; 13-[(3-O-β-D-glucopyranosyl-β-D-glucopyranosyl)oxy] kaur-16-en-18-oic acid P-D-glucopyranosyl ester; 13-[(2-O-6-deoxy-β-D-glucopyranosyl-3-O-β-D-glucopyranosyl-β-D-glucopyranosyl)oxy] kaur-16-en-18-oic acid β-D-glucopyranosyl ester; 13-[(2-O-6-deoxy β-D-glucopyranosyl-β-D-glucopyranosyl)oxy] kaur-16-en-18-oic acid β-D-glucopyranosyl ester; and mixtures thereof.

Additional illustrative sweetness enhancers include rebaudioside C, rebaudioside F, rebaudioside D, 13-[(2-O-β-D-glucopyranosyl-3-O-β-D-glucopyranosyl]-β-D-glucopyranosyl)oxyl-17-hydroxy-kaur-15-en-18-oic acid β-D-glucopyranosyl ester, 13-[(2-O-(3-O-β-D-glucopyranosyl)-β-D-glucopyranosyl-3-O-β-D-glucopyranosyl-β-D-glucopyranosyl)oxy] kaur-16-en-18-oic acid β-D-glucopyranosyl ester, and Rubusoside. Further for example, the at least one sweetness enhancer is chosen from rebaudioside A, stevioside, rebaudioside D, rebaudioside E, mogroside V, mogroside IV, brazzein, and monatin. In some embodiments, the taste-enhancing compounds disclosed herein are present in the flavored article in combination with a rebaudioside, sucralose, a mogroside, or any combination thereof.

In some embodiments, the at least one sweetness enhancer is present in an amount at or below the sweetness detection threshold level of the at least one sweetness enhancer. In some aspects, the at least one sweetness enhancer is present in an amount below the sweetness detection threshold level of the at least one sweetness enhancer. The sweetness detection threshold level can be specific for a particular compound. However, generally, in some aspects, the at least one sweetness enhancer is present in an amount ranging from 0.5 ppm to 1000 ppm. For example, the at least one sweetness enhancer may be present in an amount ranging from 1 ppm to 300 ppm; and at least one sweetness enhancer may be present in an amount ranging from 0.1 ppm to 75 ppm; and at least one sweetness enhancer may be present in an amount ranging from 500 ppm to 3,000 ppm.

As used herein, the terms “sweetness threshold,” “sweetness recognition threshold,” and “sweetness detection threshold” are understood to mean the level at which the lowest known concentration of a certain sweet compound that is perceivable by the human sense of taste and it can vary from person to person. For example, a typical sweetness threshold level for sucrose in water can be 0.5%. Further for example, the at least one sweetness enhancer to be used can be assayed in water at least 25% lower and at least 25% higher than the sucrose detection level of 0.5% in water to determine the sweetness threshold level. A person of skill in the art will be able to select the concentration of the at least one sweetness enhancer so that it may impart an enhanced sweetness to a composition comprising at least one sweetener. For example, a skilled artisan may select a concentration for the at least one sweetness enhancer so that the at least one sweetness enhancer does not impart any perceptible sweetness to a composition that does not comprise at least one sweetener. In some embodiments, the compounds listed above as sweeteners may also function as sweetness enhancers. Generally speaking, some sweeteners may also function as sweetness enhancers and vice versa.

### Formulations

In some embodiments, the present disclosure provides formulations comprising the composition according to several aspects presented herein. In these formulations, the composition according to several aspects presented herein may take any suitable form including, but not limited to, an amorphous solid, a crystal, a powder, a tablet, a liquid, a cube, a glace or coating, a granulated product, an encapsulated form abound to or coated on to carriers/particles, wet or dried, or combinations thereof.

For example, in one embodiment, the composition according to several aspects presented herein can be provided in pre-portioned packets or ready-to-use formulations, which include a composition comprising the at least one molecule comprising a monoester derivative of a primary alcohol residue of a sugar.

In some embodiments, the formulation set forth herein contain further additives known to those skilled in the art. These additives include but are not limited to bubble forming agents, bulking agents, carriers, fibers, sugar alcohols, oligosaccharides, sugars, high intensity sweeteners, nutritive sweeteners, flavorings, flavor enhancers, flavor stabilizers, acidulants, anti-caking and free-flow agents. Such additives are for example described by H. Mitchell, Sweeteners and Sugar Alternatives in Food Technology (2006), which is incorporated herein by reference in its entirety. As used herein, the term “flavorings” may include those flavors known to the skilled person, such as natural and artificial flavors. These flavorings may be chosen from synthetic flavor oils and flavoring aromatics or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

In some embodiments, the flavoring may be employed in either liquid form or dried form. When employed in the latter form, suitable drying means such as spray drying the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In some aspects, the flavorings may be used in many distinct physical forms well-known in the art to provide an initial burst of flavor or a prolonged sensation of flavor. Without being limited thereto, such physical forms include free forms, such as spray dried, powdered, beaded forms, encapsulated forms, and mixtures thereof.

In some aspects, the present disclosure provides a table-top sweetener product comprising the composition according to several aspects presented herein.

The term “table-top sweetener,” as used herein, refers to sweetener compositions that comprise at least one sweetener, and optionally, at least one sweetness enhancer, which can be used in the preparation of various food items or as an additive to food items. As one example, the tabletop sweetener may be used in the preparation of baked goods or other sweetened foods. As another example, the tabletop sweetener may be used to season, sweeten, or otherwise customize a prepared food item, e.g., beverages, fruit, or yoghurt.

In one aspect, the tabletop sweetener is in a crystalline, granulated, or powder form. In various aspects, the tabletop sweetener may comprise one or more sweeteners or one or more sweetness enhancers. In one aspect, the tabletop sweetener may comprise either or both a caloric sweetener or substantially non-caloric sweeteners, and, if appropriate, one or more sweetness enhancers. Typical examples of caloric sweeteners that may be used in tabletop sweeteners include sucrose, fructose, and glucose. Common tabletop forms of these caloric sweeteners include cane sugar, bee sugar, and the like. In recent decades, substantially non-caloric sweeteners have gained popularity. In many instances, these sweeteners can be used as substitutes for caloric sweeteners and are often referred to as “sugar substitutes.”

Illustrative table-top sweetener products are disclosed in PCT Publication No. WO2012/107203.

In some embodiments, the table-top sweetener product comprises an effective amount of a composition comprising the at least one molecule comprising a monoester derivative of a primary alcohol residue of a sugar.

In some embodiments, the present disclosure provides a flavored article comprising the composition according to several aspects presented herein. In some aspects, the flavored article comprises an olfactory or gustatory effective amount of a composition comprising the at least one molecule comprising a monoester derivative of a primary alcohol residue of a sugar.

Flavored articles include, but are not limited to beverages, dental products, cosmetic products, pharmaceutical products and animal feed or animal food. For example, consumable products include all food products, including but not limited to cereal products, rice products, tapioca products, sago products, baker's products, biscuit products, pastry products, bread products, confectionary products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cacoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof.

As used herein, the term “non-alcoholic drinks” includes, but is not limited to all nonalcoholic drinks mentioned in the Directive 2003/115/EC, 22 Dec. 2003, and in the Directive 94/35/EC, 30 Jun. 2004, which are incorporated herein by reference, on sweeteners for use in foodstuffs. Examples include, but are not limited to water-based, flavored drinks, energy-reduced or with no added sugar, milk- and milk-derivative-based or fruit-juice-based drinks, energy-reduced or with no added sugar, “Gaseosa”: nonalcoholic water-based drink with added carbon dioxide, sweeteners and flavorings.

Flavored articles include without limitation, water-based consumables, solid dry consumables, dairy products, dairy-derived products and dairy-alternative products. In one aspect, the consumable product is a water-based consumable product including but not limited to beverage, water, aqueous beverage, enhanced/slightly sweetened water drink, flavored carbonated and still mineral and table water, carbonated beverage, non-carbonated beverage, carbonated water, still water, soft drink, non-alcoholic drink, alcoholic drink, beer, wine, liquor, fruit drink, juice, fruit juice, vegetable juice, broth drink, coffee, tea, black tea, green tea, oolong tea, herbal infusion, cacao (e.g. water-based), tea-based drink, coffee-based drinks, cacao-based drink, infusion, syrup, frozen fruit, frozen fruit juice, water-based ice, fruit ice, sorbet, dressing, salad dressing, jams, marmalades, canned fruit, savoury, delicatessen products like delicatessen salads, sauces, ketchup, mustard, pickles and marinated fish, sauce, soup, and beverage botanical materials (e.g. whole or ground), or instant powder for reconstitution (e.g. coffee beans, ground coffee, instant coffee, cacao beans, cacao powder, instant cacao, tea leaves, instant tea powder). In another embodiment, the consumable product is a solid dry consumable product including but not limited to cereals, baked food products, biscuits, bread, breakfast cereal, cereal bar, energy bars/nutritional bars, granola, cakes, rice cakes, cookies, crackers, donuts, muffins, pastries, confectionaries, chewing gum, chocolate products, chocolate, fondant, hard candy, marshmallow, pressed tablets, snack foods, botanical materials (whole or ground), and instant powders for reconstitution.

The present invention is best illustrated but is not limited to the following examples.

### EXAMPLES

### Example 1

### Synthesis of Flavor-Enhancing Compounds

Synthesis of glucose-6-O-acetate: 3 g glucose, 150 ml tert-butanol, 19.2 g acetic acid, 6 g of a 4 Å molecular sieve (Alfa Aesar, Karlsruhe, Germany), and 3 g of a lipase consisting of an immobilized lipase, obtained from Candida antartica, sold under the trade name NOVOZYM 435 were added to a 500 ml Erlenmeyer flask. The reaction mixture was incubated at 50° C. for 4 days in an orbital shaker.

Next, the reaction mixture was filtered over a cotton plug, rinsed with 50 to 100 ml ethanol and concentrated to obtain a concentrated reaction product. The concentrated reaction product was dissolved in ethyl acetate and applied to a silica column (19×5.5 cm I.D.). Unreacted acetic acid in the concentrated reaction product was removed by washing the silica column with ethyl acetate. Glucose-6-O-acetate was eluted off the column by applying a 1:1 ratio of ethyl acetate/ethanol to the silica column, until no further glucose-6-O-acetate was detected coming off the column. The eluted glucose-6-O-acetate was then concentrated, dissolved in water, freeze dried and stored at room temperature.

Synthesis of glucose-6-O-hexanoate: 3 g glucose, 150 ml tert-butanol, 19.2 g hexanoic acid, 6 g of a 4 Å molecular sieve (Alfa Aesar, Karlsruhe, Germany), and 3 g of a lipase consisting of an immobilized lipase, obtained from Candida antartica, sold under the trade name NOVOZYM 435 were added to a 500 ml Erlenmeyer flask. The reaction mixture was incubated at 50° C. for 4 days in an orbital shaker.

Next, the reaction mixture was filtered, rinsed with 50 to 100 ml ethanol and concentrated to obtain a concentrated reaction product. The concentrated reaction product was dissolved in ethyl acetate and applied to a silica column (19×5.5 cm I.D.). Unreacted hexanoic acid in the concentrated reaction product was removed by washing the silica column with ethyl acetate. Glucose-6-O-hexanoate was eluted off the column by applying a 1:1 ratio of ethyl acetate/ethanol to the silica column, until no further glucose-6-O-hexanoate was detected coming off the column. The eluted glucose-6-O-hexanoate was then concentrated, dissolved in water, freeze dried and stored at room temperature.

Synthesis of glucose-6-O-octanoate: 3 g glucose, 150 ml tert-butanol, 19.2 g octanoic acid, 6 g of a 4 Å molecular sieve (Alfa Aesar, Karlsruhe, Germany), and 3 g of a lipase consisting of an immobilized lipase, obtained from Candida antartica, sold under the trade name NOVOZYM 435 were added to a 500 ml Erlenmeyer flask. The reaction mixture was incubated at 50° C. for 4 days in an orbital shaker.

Next, the reaction mixture was filtered, rinsed with 50 to 100 ml ethanol and concentrated to obtain a concentrated reaction product. The concentrated reaction product was dissolved in ethyl acetate and applied to a silica column (19×5.5 cm I.D.). Unreacted octanoic acid in the concentrated reaction product was removed by washing the silica column with ethyl acetate. Glucose-6-O-octanoate was eluted off the column by applying a 1:1 ratio of ethyl acetate/ethanol to the silica column, until no further glucose-6-O-octanoate was detected coming off the column. The eluted glucose-6-O-octanoate was then concentrated, dissolved in water, freeze dried and stored at room temperature.

The reaction outlined above was repeated using the following acid donor molecules: acetic acid, hexanoic acid, decanoic acid, lauric acid, myristic acid, palmitic acid, oleic acid, butter oil, olive oil, phenylpropanoic acid, and cinnamic acid. Typical yields are reported in Table 1 below.

TABLE 1

| R-COOH (acid | Chain | Yield |
|---|---|---|
| donor) | length | % |
| Octanoid acid | C_{8:0} | 52- |
| | | 90 |
| Acetic acid | C_{2:0} | 40 |
| Hexanoic acid | C_{6:0} | 62 |
| Decanoic acid | C₁₀₀ | 49 |
| Lauric acid | C₁₂₀ | 20 |
| Myristic acid | C_{14:0} | 35 |
| Palmitic acid | C₁₆₀ | 21 |
| Oleic acid | C₁₈₁ (9-Z) | 57 |
| Butter oil | various | 39^{a} |
| Olive oil | C_{18:1} main | 49 |
| 3-Phenylpropanoic | C₉ | 40 |
| Cinnamic acid | C₉ | 31 |

The reaction outlined above was also repeated using fructose as the sugar, and octanoic acid as the acid donor molecule. The enzymatic esterification of D-(−)-fructose gave a complex mixture: Besides diesters, 6-O-octanoyl-β-fructofuranose, 1-O-octanoyl-α-fructopyranose and 1-O-octanoyl-β-fructofuranose were identified in the synthetic mixture after isolation, by preparative HPLC.

¹³C NMR were recorded for certain of the synthesized compounds. Table 2 shows the recorded chemical shifts for ¹³C NMR for the respective compounds.

TABLE 2

| Compound | Recorded Proton NMR Chemical Shifts |
|---|---|
| Glucose- | ¹³C NMR (150 MHz, MeOD): δ 14.4 (q), 23.7 (t), 26.1 (t), 30.1 |
| 6-O- | (t), 30.2 (t), 32.9 (t), 35.0 (t), 64.8 (t), 70.7 (d), 72.0 (d), 73.8 (d), |
| octanoate | 74.8 (d), 94.0 (d), 175.6 (s) |
| Glucose- | ¹³C NMR (150 MHz, MeOD): δ 14.3 (q), 23.4 (t), 25.8 (t), 32.4 |
| 6-O- | (t), 35.0 (t), 64.8 (t), 70.7 (d), 72.0 (d), 73.8 (d), 74.8 (d), 94.0 |
| hexanoate | (d), 175.6 (s) |
| Glucose- | ¹³C NMR (150 MHz, MeOD): δ 14.5 (q), 23.8 (t), 26.1 (t), 30.2 |
| 6-O- | (t), 30.4 (t), 30.5 (t), 30.6 (t), 33.1 (t), 35.0 (t), 64.8 (t), 70.7 (d), |
| decanoate | 72.0 (d), 73.8 (d), 74.8 (d), 94.0 (d), 175.6 (s) |
| Glucose- | ¹³C NMR (150 MHz, MeOD): δ 14.5 (q), 23.8 (t), 26.1 (t), 30.2 |
| 6-O- | (t), 30.5 (t), 30.5 (t), 30.5 (t), 30.7 (t), 30.8 (t), 33.1(t), 35.0 (t), |
| laurate | 64.8 (t), 70.7 (d), 72.0 (d), 73.8 (d), 74.8 (d), 94.0 (d), 175.6 (s) |
| Glucose- | ¹³C NMR (150 MHz, MeOD): δ 14.5 (q); 23.8 (t); 26.1 (t); 30.2 |
| 6-O- | (t); 30.5 (t); 30.5 (t); 30.5 (t); 30.7 (t); 30.8 (t); 30.8 (t); 30.8 (t); |
| palmitate | 30.8 (t); 30.8 (t); 33.1 (t); 35.0 (t); 64.8 (t); 70.7 (d); 72.0 (d); |
| | 73.8 (d); 74.8 (d); 94.0 (d); 175.6 (s) |
| Glucose- | ¹³C NMR (150 MHz, MeOD): δ 14.5 (q), 23.8 (t), 26.0 (t), 28.1 |
| 6-O- | (t), 30.2 (t), 30.2 (t), 30.3 (t), 30.4 (t), 30.5 (t), 30.6 (t), 30.8 (t), |
| oleate | 30.9 (t), 33.1 (t), 35.0 (t), 64.8 (t), 70.7 (d), 72.0 (d), 73.8 (d), |
| | 74.8 (d), 94.0 (d), 130.8 (d), 130.9 (d), 175.5 (s) |

### Example 2

### Sensory Properties

Glucose-6-O-octanoate, manufactured according to the methods described in Example 1 above was added to a dairy test composition (milk sweetened with 3% w/v sucrose), to result in a final concentration of 100 ppm in the test composition. In parallel, a control composition, comprising milk sweetened with 3% w/v sucrose was also generated. A sensory panel consisting of between 6 to 8 persons compared the overall flavor, sweetness, acidity, bitterness, astringency, off-notes, and long-lastingness of the sweetness of the dairy test composition containing 100 ppm glucose-6-O-octanoate and the control composition. The results were analyzed using the Duncan comparison test.

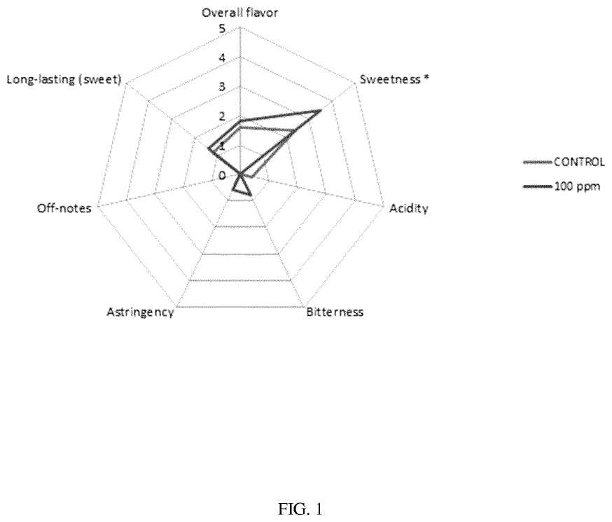

The results are shown in FIG. 1 and Table 3 below. Key: Results are significantly different at 90%, * at 95%, ** at 99% and *** at 99.9% of confidence from the Duncan comparison test. Samples having the same letter were not significantly different according to the Duncan comparison test.

TABLE 3

| | Sweetened | Sweetened milk | | |
|---|---|---|---|---|
| | milk | 3%-100 ppm | | |
| | 3%- | glucose-6-O- | F | |
| Analysis | CONTROL | octanoate | Calc. | Proba. |
| Overall | 1.6 | 1.8 | 1.00 | 0.3739 |
| Flavor | | | | |
| Sweetness | 2.4 B | 3.5 A | 7.56 | 0.0514 |
| | | | | ! |
| Acidity | 0.4 | 0 | 2.67 | 0.1778 |
| Bitterness | 0 | 0.8 | 1.00 | 0.3739 |
| Astringency | 0.4 | 0.6 | 0.17 | 0.7040 |
| Long- | 1.2 | 1.4 | 1.00 | 0.3739 |
| Lasting | | | | |

These data suggest that 100 ppm glucose-6-O-octanoate is capable of enhancing the perceived sweetness of the dairy test formulation.

Next, glucose-6-O-octanoate was added to a coffee composition (Emmi Noir coffee, containing milk), to result in a final concentration of 200 ppm in the test composition. In parallel, a control composition, comprising Emmi Noir coffee, containing milk was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

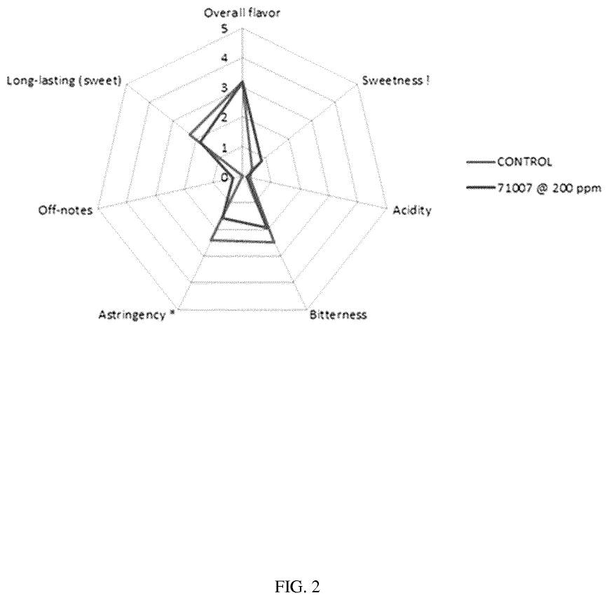

The results are shown in FIG. 2 and Table 4 below. Key: Results are significantly different at 90%, * at 95%, ** at 99% and *** at 99.9% of confidence from the Duncan comparison test. Samples having the same letter were not significantly different according to the Duncan comparison test.

TABLE 4

| | | glucose- | | |
|---|---|---|---|---|
| | | 6-O- | | |
| | | octanoate | | |
| | | @200 | F | |
| Analysis | CONTROL | ppm | Calc. | Proba. |
| Overall flavor | 3.17 | 3.17 | 0.00 | 1.0000 |
| Sweetness | 0.42 B | 0.83 A | 4.31 | 0.0925 |
| | | | | ! |
| Acidity | 0.25 | 0.17 | 1.00 | 0.3632 |
| Bitterness | 2.5 | 1.92 | 2.43 | 0.1801 |
| Astringency | 2.42 A | 1.58 B | 7.35 | 0.0422 |
| | | | | * |
| Off-notes | 0 | 0.33 | 1.00 | 0.3632 |
| Long-lasting | 2.25 | 1.83 | 1.62 | 0.2586 |
| (sweet) | | | | |

These data suggest that 200 ppm glucose-6-O-octanoate is capable of enhancing the perceived sweetness and decreasing the astringency of the coffee drink test formulation.

Next, glucose-6-O-octanoate was added to a fruit flavored bottled water test composition (strawberry flavored water, containing 7% w/v sucrose), to result in a final concentration of 100 ppm in the test composition. In parallel, a control composition, comprising (strawberry flavored water, containing 7% w/v sucrose was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

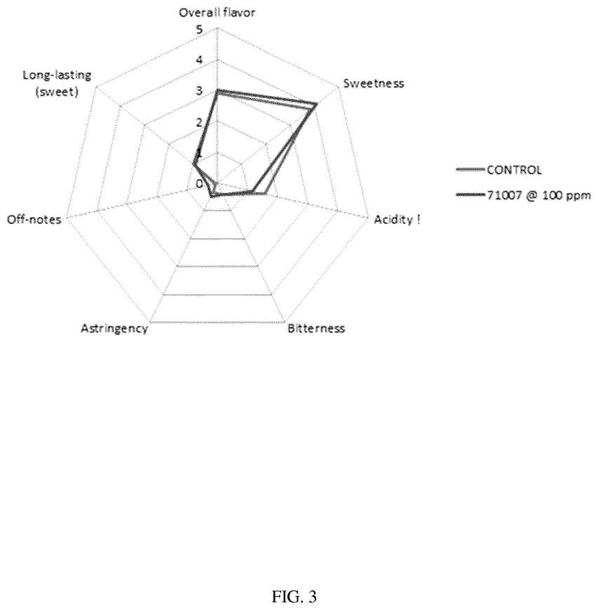

The results are shown in FIG. 3 and Table 5 below. Key: Results are significantly different at 90%, * at 95%, ** at 99% and *** at 99.9% of confidence from the Duncan comparison test. Samples having the same letter were not significantly different according to the Duncan comparison test.

TABLE 5

| | | BWAT 7% | | |
|---|---|---|---|---|
| | BWAT | Sucrose- | | |
| | 7% | 100 ppm | | |
| | Sucrose- | glucose- | F | |
| Analysis | CONTROL | 6-O-octanoate | Calc. | Proba. |
| Overall | 2.92 | 3 | 1.00 | 0.3632 |
| Flavor | | | | |
| Sweetness | 3.83 | 4.08 | 2.14 | 0.2031 |
| Acidity | 1.58 A | 1.17 B | 4.31 | 0.0925 |
| | | | | ! |
| Astringency | 0.33 | 0.5 | 2.50 | 0.1747 |
| Off-notes | 0.08 | 0.33 | 1.00 | 0.3632 |
| Long-Lasting | 1 | 0.92 | 0.03 | 0.8797 |

These data suggest that 100 ppm glucose-6-O-octanoate is capable of decreasing the perceived acidity of the strawberry flavored bottled water test formulation.

Next, glucose-6-O-octanoate was added to a lemon flavored alcoholic beverage test formulation to result in a final concentration of 100 ppm in the test composition. In parallel, a control composition, comprising lemon flavored alcoholic beverage was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

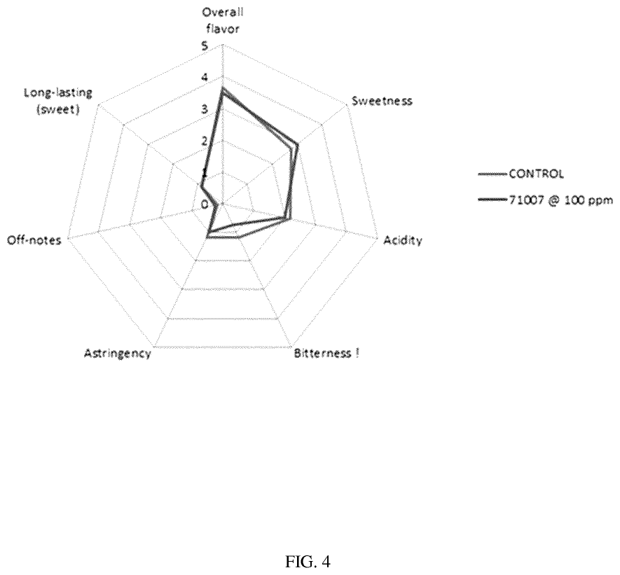

The results are shown in FIG. 4 and Table 6 below. Key: Results are significantly different at 90%, * at 95%, ** at 99% and *** at 99.9% of confidence from the Duncan comparison test. Samples having the same letter were not significantly different according to the Duncan comparison test.

TABLE 6

| | | glucose-6-O- | | |
|---|---|---|---|---|
| | | octanoate | | |
| | | @100 | F | |
| Analysis | CONTROL | ppm | Calc. | Proba. |
| Overall | 3.67 | 3.5 | 1.00 | 0.3632 |
| Flavor | | | | |
| Sweetness | 2.75 | 3 | 2.14 | 0.2031 |
| Acidity | 2.17 | 2 | 1.00 | 0.3632 |
| Bitterness | 1.17 A | 0.75 B | 4.31 | 0.0925 |
| | | | | ! |
| Astringency | 1.17 | 1 | 1.00 | 0.3632 |
| Off-notes | 0.17 | 0.25 | 1.00 | 0.3632 |

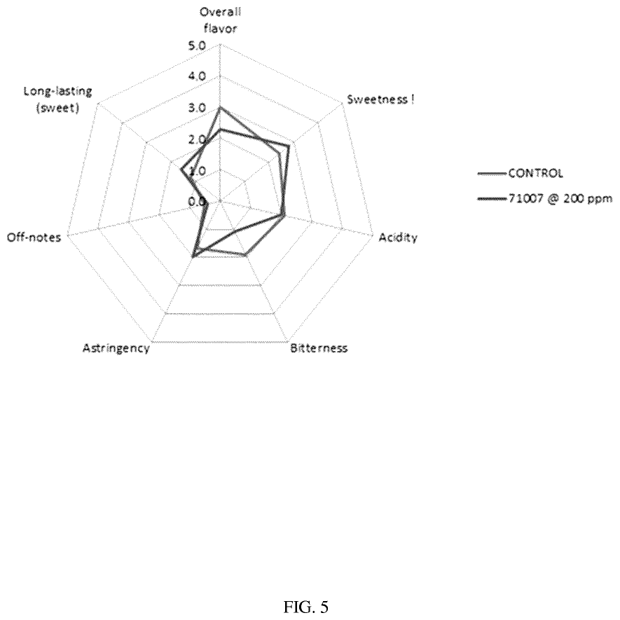

These data suggest that 100 ppm glucose-6-O-octanoate is capable of decreasing the perceived bitterness of the lemon flavored alcoholic beverage test formulation. Referring to FIG. 5, and Table 7 below, increasing the concentration of glucose-6-O-octanoate in the lemon flavored alcoholic beverage test formulation to 200 ppm appeared to increase the perceived sweetness of the lemon flavored alcoholic beverage test formulation.

TABLE 7

| | | glucose- | | |
|---|---|---|---|---|
| | | 6-O- | | |
| | | octanoate | | |
| | | @200 | F | |
| Analysis | CONTROL | ppm | Calc. | Proba. |
| Overall | 3 | 2.3 | 1.26 | 0.3251 |
| Flavor | | | | |
| Sweetness | 2.4 B | 2.8 A | 4.57 | 0.0993 |
| | | | | ! |
| Acidity | 2.1 | 2 | 0.09 | 0.7780 |
| Bitterness | 1.9 | 1.1 | 0.79 | 0.4256 |
| Astringency | 1.7 | 2 | 0.23 | 0.6560 |
| Off-notes | 0.5 | 0.4 | 1.00 | 0.3739 |
| Long-Lasting | 1.2 | 1.6 | 2.67 | 0.1778 |

Next, glucose-6-O-octanoate was added to a pomegranate juice composition, to result in a final concentration of 100 ppm in the test composition. In parallel, a control composition, comprising pomegranate juice was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

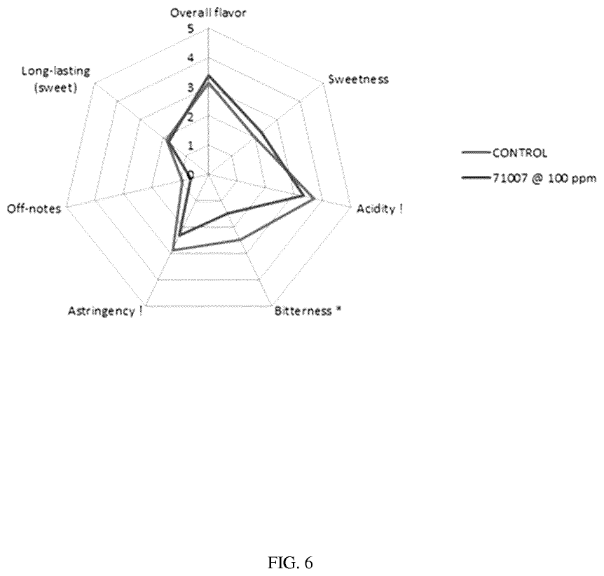

The results are shown in FIG. 6 and Table 8 below. Key: Results are significantly different at 90%, * at 95%, ** at 99% and *** at 99.9% of confidence from the Duncan comparison test. Samples having the same letter were not significantly different according to the Duncan comparison test.

TABLE 8

| | | glucose-6-O- | | |
|---|---|---|---|---|
| | | octanoate @ | F | |
| Analysis | CONTROL | 100 ppm | Calc. | Proba. |
| Overall Flavor | 3.12 | 3.38 | 2.33 | 0.1705 |
| Sweetness | 2 | 2.31 | 0.55 | 0.4830 |
| Acidity | 3.69 A | 3.31 B | 4.20 | 0.0796 |
| | | | | ! |
| Bitterness | 2.5 A | 1.5 B | 7.00 | 0.0331 |
| | | | | * |
| Astringency | 2.88 A | 2.31 B | 4.76 | 0.0654 |
| | | | | ! |
| Off-notes | 0.94 | 0.62 | 1.58 | 0.2495 |
| Long-Lasting | 1.87 | 1.75 | 1.00 | 0.3506 |

These data suggest that 100 ppm glucose-6-O-octanoate is capable of enhancing the perceived sweetness and decreasing the astringency, bitterness and acidity of the pomegranate juice test formulation.

Next, glucose-6-O-octanoate was added to an energy drink formulation to result in a final concentration of 100 ppm in the test composition. In parallel, a control energy drink composition was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

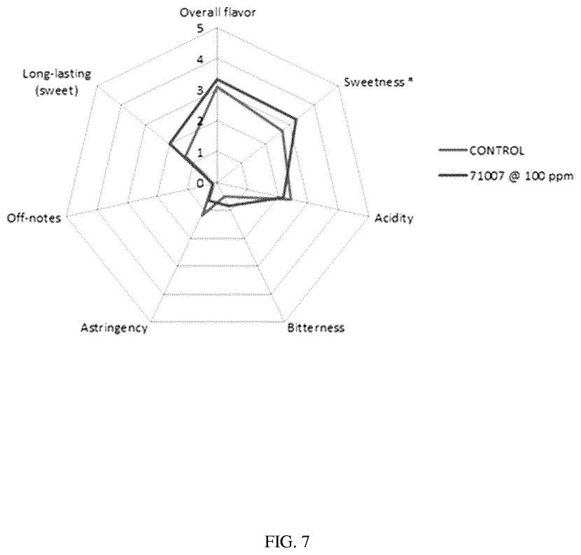

The results are shown in FIG. 7 and Table 9 below. Key: Results are significantly different at 90%, * at 95%, ** at 99% and *** at 99.9% of confidence from the Duncan comparison test. Samples having the same letter were not significantly different according to the Duncan comparison test.

TABLE 9

| | | glucose- | | |
|---|---|---|---|---|
| | | 6-O- | | |
| | | octanoate @ | F | |
| Analysis | CONTROL | 100 ppm | Calc. | Proba. |
| Overall | 3.08 | 3.33 | 1.00 | 0.3632 |
| Flavor | | | | |
| Sweetness | 2.67 B | 3.25 A | 8.45 | 0.0335 |
| | | | | * |
| Acidity | 2.42 | 2.17 | 2.14 | 0.2031 |
| Bitterness | 0.5 | 0.83 | 1.00 | 0.3632 |
| Astringency | 1.17 | 0.67 | 2.14 | 0.2031 |
| Long-Lasting | 1.33 | 2 | 2.76 | 0.1576 |

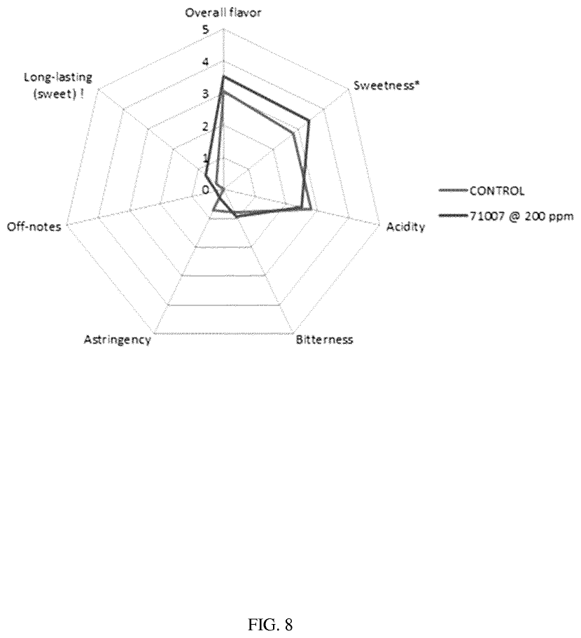

These data suggest that 100 ppm glucose-6-O-octanoate is capable of increasing the perceived sweetness of the energy drink test formulation. Referring to FIG. 8, and Table 10 below, increasing the concentration of glucose-6-O-octanoate in the energy drink test formulation to 200 ppm appeared to increase the perceived sweetness and long-lastingness of the perceived sweetness of the energy drink test formulation.

TABLE 10

| | | glucose- | | |
|---|---|---|---|---|
| | | 6-O- | | |
| | | octanoate | | |
| | | @200 | F | |
| Analysis | CONTROL | ppm | Calc. | Proba. |
| Overall | 3.07 | 3.5 | 1.64 | 0.2481 |
| Flavor | | | | |
| Sweetness | 2.79 B | 3.43 A | 9.35 | 0.0223 |
| | | | | * |
| Acidity | 2.79 | 2.5 | 0.63 | 0.4571 |
| Bitterness | 0.79 | 0.93 | 0.20 | 0.6729 |
| Astringency | 0.71 | 0.29 | 0.58 | 0.4738 |
| Off-notes | 0 | 0.21 | 2.08 | 0.1996 |
| Long-Lasting | 0.29 B | 0.71 A | 4.50 | 0.781 ! |

Next, glucose-6-O-octanoate was added to a raspberry flavored water formulation to result in a final concentration of 100 ppm in the test composition. In parallel, a control composition, comprising raspberry flavored water was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

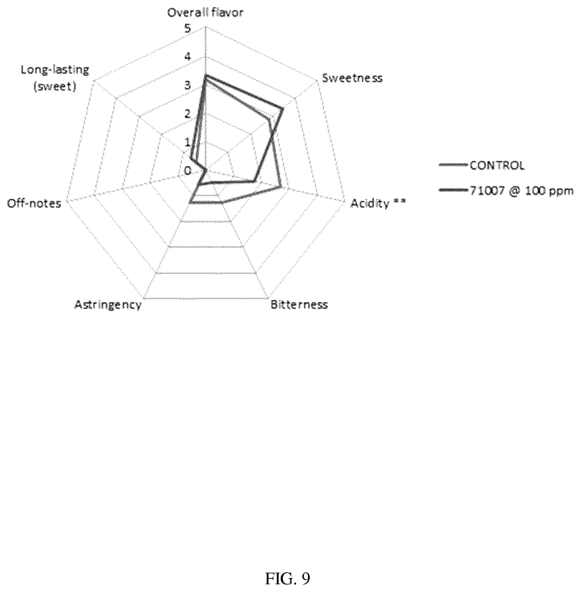

The results are shown in FIG. 9 and Table 11 below. Key: Results are significantly different at 90%, * at 95%, ** at 99% and *** at 99.9% of confidence from the Duncan comparison test. Samples having the same letter were not significantly different according to the Duncan comparison test.

TABLE 11

| | | Raspberry | | |
|---|---|---|---|---|
| | | Water- | | |
| | | glucose- | | |
| | Raspberry | 6-O- | | |
| | Water- | octanoate @ | F | |
| Analysis | CONTROL | 100 ppm | Calc. | Proba. |
| Overall | 3.17 | 3.33 | 1.00 | 0.3632 |
| Flavor | | | | |
| Sweetness | 2.83 | 3.42 | 3.18 | 0.1345 |
| Acidity | 2.67 A | 1.75 B | 11.42 | 0.0197 |
| | | | | ** |
| Bitterness | 1.25 | 0.5 | 2.45 | 0.1780 |
| Astringency | 1.25 | 0.58 | 4.00 | 0.1019 |
| Long-Lasting | 0.42 | 0.67 | 2.14 | 0.2031 |

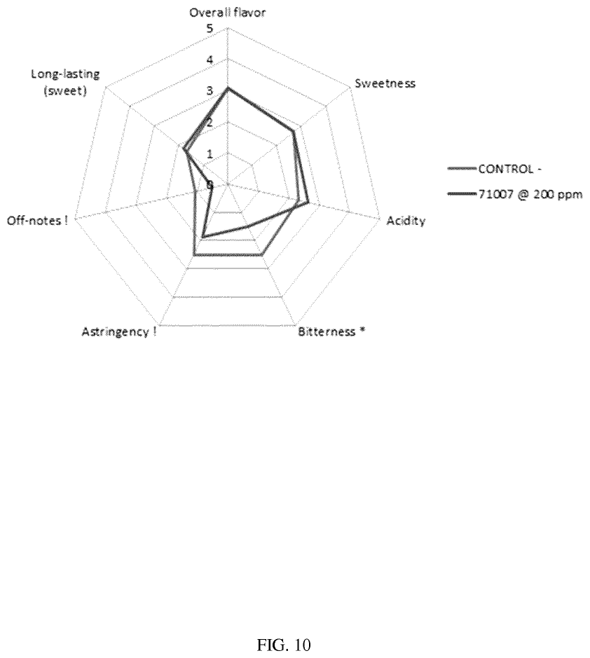

These data suggest that 100 ppm glucose-6-O-octanoate is capable of decreasing the perceived acidity of the raspberry flavored water test formulation. Referring to FIG. 10, and Table 12 below, increasing the concentration of glucose-6-O-octanoate in the raspberry flavored water test formulation to 200 ppm appeared to decrease the perceived bitterness, astringency and off-notes of the raspberry flavored water test formulation.

TABLE 12

| | | glucose- | | |
|---|---|---|---|---|
| | | 6-O- | | |
| | | octanoate | | |
| | | @200 | F | |
| Analysis | CONTROL | ppm | Calc. | Proba. |
| Overall | 3.06 | 3.06 | 0.00 | 1.0000 |
| Flavor | | | | |
| Sweetness | 2.69 | 2.69 | 0.00 | 1.0000 |
| Acidity | 2.31 | 2.62 | 0.85 | 0.3884 |
| Bitterness | 2.5 A | 1.5 B | 7.00 | 0.0331 |
| | | | | * |
| Astringency | 2.5 A | 1.88 B | 5.00 | 0.0604 |
| | | | | ! |
| Off-notes | 1.06 A | 0.5 B | 4.76 | 0.0654 |
| | | | | ! |
| Long-Lasting | 1.69 | 1.81 | 1.00 | 0.3506 |

Next, glucose-6-O-octanoate was added to an orange flavored test composition, to result in a final concentration of 200 ppm in the test composition. In parallel, a control composition, comprising orange flavor was also generated. A sensory panel consisting of between 6 to 8 persons compared the overall flavor, sweetness, acidity, bitterness, astringency, off-notes, and long-lastingness of the sweetness of the test composition and the control composition. The results were analyzed using the Duncan comparison test.

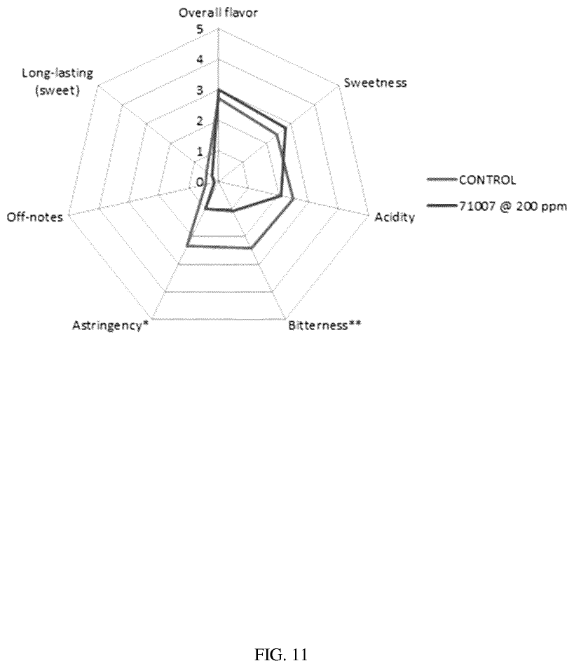

The results are shown in FIG. 11 and Table 13 below. Key: Results are significantly different at 90%, * at 95%, ** at 99% and *** at 99.9% of confidence from the Duncan comparison test. Samples having the same letter were not significantly different according to the Duncan comparison test.

TABLE 13

| | | 71007 | | |
|---|---|---|---|---|
| | | @200F | F | |
| Analysis | CONTROL | ppm | Calc. | Proba. |
| Overall | 2.71 | 3 | 1.17 | 0.3208 |
| Flavor | | | | |
| Sweetness | 2.43 | 2.79 | 1.39 | 0.2832 |
| Acidity | 2.5 | 2.07 | 1.24 | 0.3078 |
| Bitterness | 2.43 A | 1.07 B | 17.75 | 0.0056 |
| | | | | ** |
| Astringency | 2.36 A | 1 B | 10.51 | 0.0176 |
| | | | | * |
| Off-notes | 0.43 | 0.14 | 1.00 | 0.3559 |
| Long-Lasting | 0.5 | 0.29 | 2.08 | 0.1996 |

These data suggest that 200 ppm glucose-6-O-octanoate is capable of decreasing the perceived bitterness and astringency of the orange flavor test formulation.

### Example 3

### Taste Modifier Properties

The sensory properties of various compositions comprising a monoester derivative of glucose were tested in various model systems, outlined in Table 14 below.

TABLE 14

| Solution | Compound | Concentration |
|---|---|---|
| Sweet | Sucrose | 4% |
| Stevia | SG95 | 0.02% |
| Bitter | paracetamol | 0.08750% |
| | quinine HCl | 0.00068% |
| Sweet/Sour | citric acid | 0.15% |
| | Sucrose | 5.00% |
| Stevia/S our | citric acid | 0.15% |
| | SG95 | 0.03% |
| Semi skimmed | semi skimmed milk | 1.5% fat |
| milk /sucrose | sucrose | 4% |
| Semi skimmed milk | semi skimmed milk | 1.5% fat |
| Grapefruit | inverted sucrose | 7% |
| model beverage | citric acid | 0.15% |
| | grapefruit flavor | 0.01% |
| | 506617 A | |

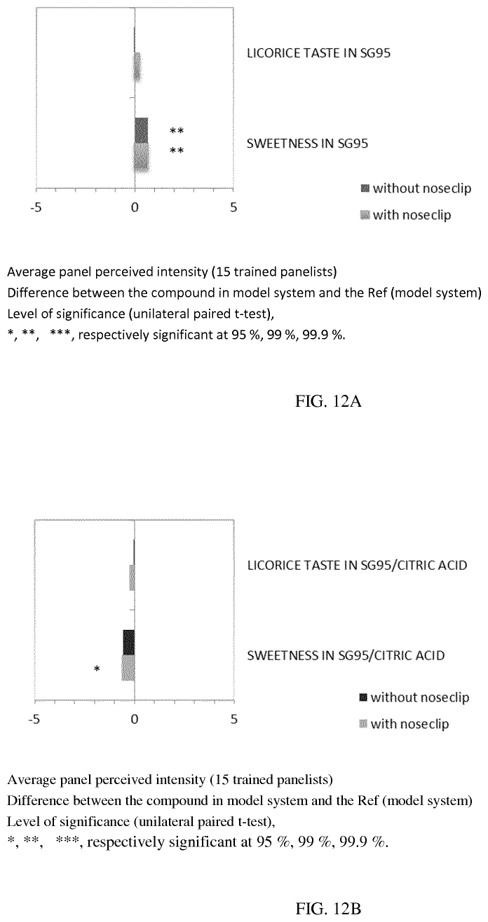

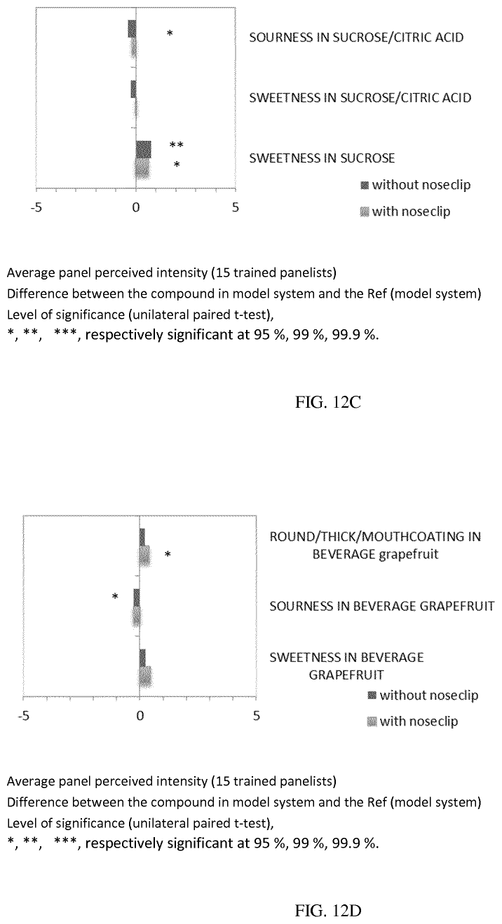

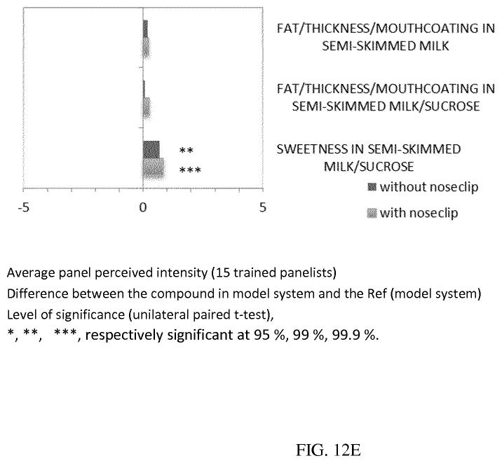

FIGS. 12 to 18 report the sweetness modifier properties of each of the following compounds (tested at 100 ppm in the various mode systems described above): glucose-6-O-octanoate, glucose-6-O-acetate, glucose-6-O-hexanoate, glucose-6-O-decanoate, glucose-6-O-hexadecanoate, glucose-6-O-oleate and glucose-6-O-oleyl derivative. The sensory properties were evaluated by a sensory panel consisting of15 persons, where the properties were evaluated with the subjects wearing a noseclip (With NC), and without a noseclip (Without NC). The subjects evaluate the perceived intensities of different sensory attributes on a linear scale (“0” note intense to “10” very intense) for the corresponding model mixtures without and with the compound at 100ppm. The differences of perceived intensities between the model mixtures with and without the compound are represented for each attribute. A statistical data treatment (unilateral paired student test) is achieved on each attribute to conclude on a significant or not significant taste modulation effect of the compound.

FIG. 12 reports the sweet modifier properties of 100ppm glucose-6-O-octanoate in various model mixtures outlined in the table above. These data suggest that at 100ppm, glucose-6-O-octanoate has significant sweet enhancing effects in sucrose, SG95 and in semi-skimmed milk+sucrose model mixtures. With a level of significance of 99% in SG95, 99% and 95% in sucrose, 99% and 99.9% in semi-skimmed milk+sucrose respectively without and with nose clip.

Glucose-6-O-octanoate has also a significant sour masking effect at 95% without nose clip in sweet-sour model mixture. It has significant sweet masking effect at 95% with nose clip in stevia-sour model. It has a significant round/thick/mouth coating enhancing effect with nose clip, and a significant sour masking effect without nose clip at 95%, in beverage grapefruit model.

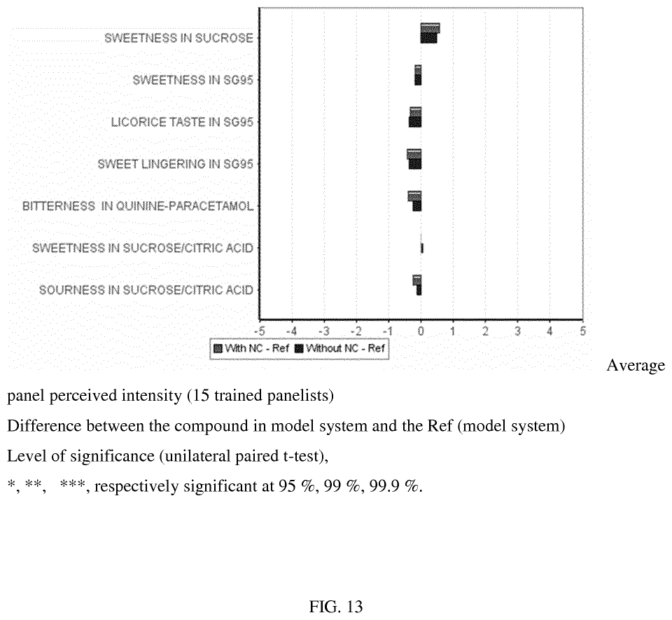

FIG. 13 reports the sweet modifier properties of 100 ppm glucose-6-O-acetate in various model mixture. These data suggest that at 100ppm, glucose-6-O-acetate had no significant modulation effects in tasted model systems. Nevertheless, glucose-6-O-acetate has some slights enhancing sweetness effects in sucrose and some slight masking effects in SG95 for licorice taste and sweet lingering. Il has also some slight bitterness masking effet.

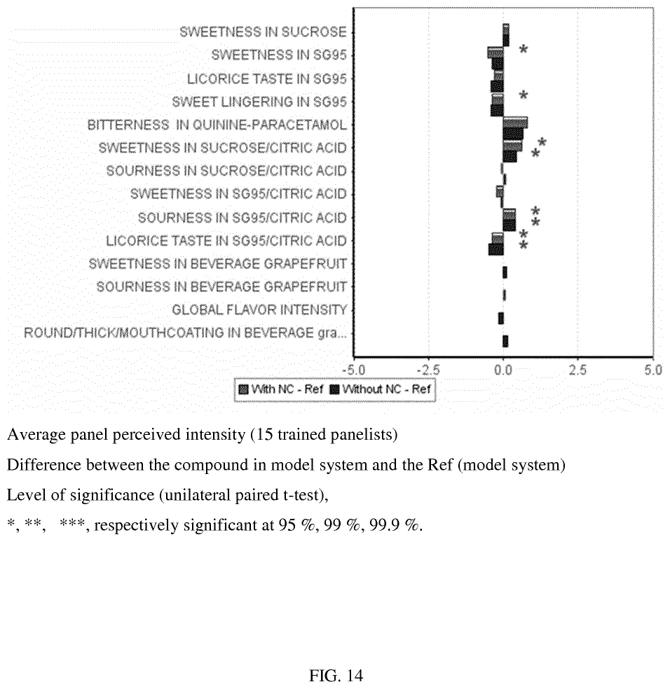

FIG. 14 reports the sweet modifier properties of 100 ppm glucose-6-O-hexanoate in various model mixtures. These data suggest that at 100ppm, glucose-6-O-hexanoate has significant sweet and sweet lingering masking effects in SG95 with nose clip at 95%. It has a significant sweet enhancing effect in sweet and sour model system with and without nose clip at 95%. Glucose-6-O-hexanoate has also a significant sour enhancing effect and a significant licorice taste masking effect in SG95-sour model system with and without nose clip at 95%.

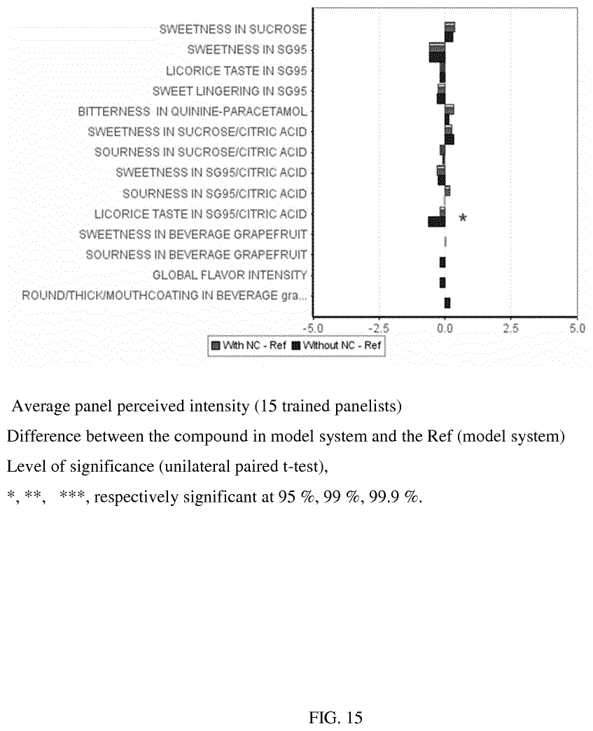

FIG. 15 reports the sweet modifier properties of 100 ppm glucose-6-O-decanoate in various model mixtures. These data suggest that at 100ppm, glucose-6-O-decanoate has a significant licorice taste masking effect in SG95-sour model system, without nose clip at 95%. There were no significant effects observed in all the other model systems.

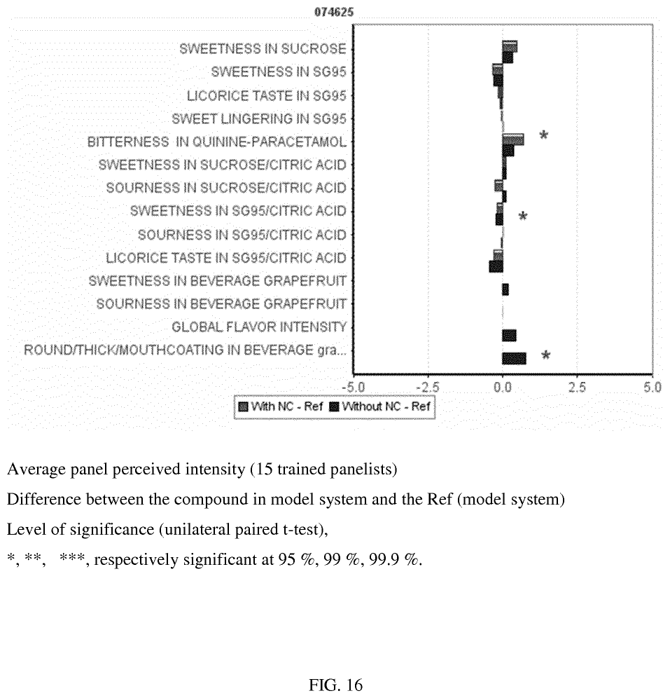

FIG. 16 reports the sweet modifier properties of 100 ppm glucose-6-O-hexadecanoate in various model mixtures. These data suggest that at 100ppm, glucose-6-O-hexadecanoate has a significant bitter enhancing effect with nose clip. It has a significant sweet masking effect in SG95/sour model system without nose clip and a significant round/thick/mouth coating enhancing effect in grapefruit beverage. All these effects are significant at a level of 95%.

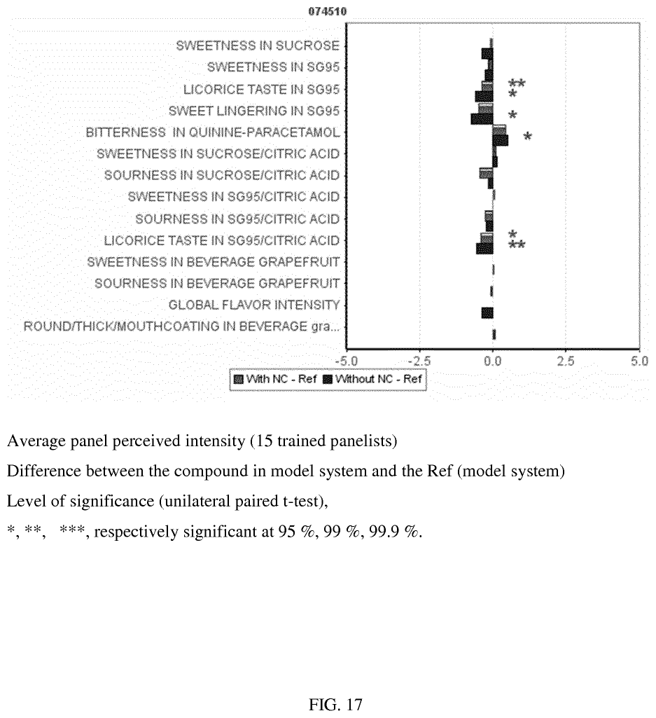

FIG. 17 reports the sweet modifier properties of glucose-6-O-oleate in various model mixtures. These data suggest that at 100ppm, glucose-6-O-oleate has a significant licorice taste masking effect in SG95 and in SG95-sour model systems, with and without nose clip at least at 95% of significance level. It has a significant bitter enhancing effect without nose clip at 95%. It has also a significant sweet lingering masking effect without nose clip at 95%.

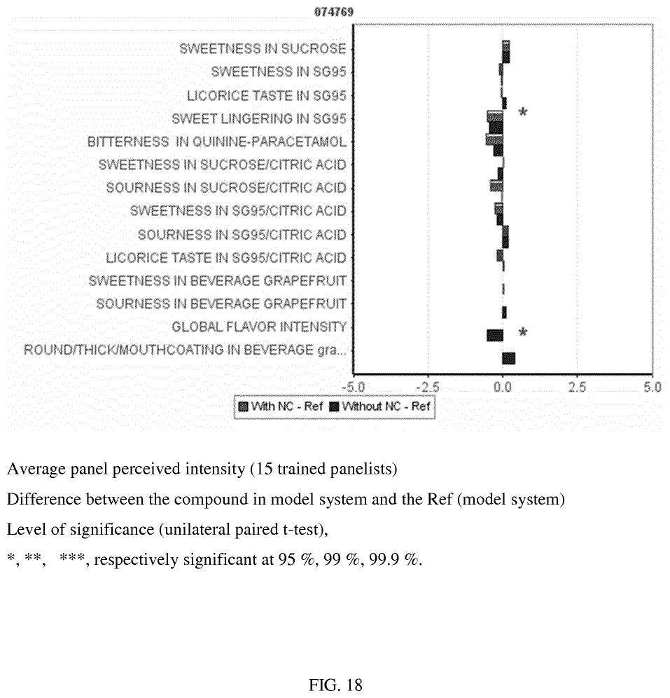

FIG. 18 reports the sweet modifier properties of 100 ppm glucose-6-O-oleyl derivative in various model mixtures. These data suggest that at 100ppm, glucose-6-O-oleyl derivative has a significant sweet lingering masking effect in SG95 model system with nose clip at 95%. It has a significant flavor intensity masking effect in beverage grapefruit at 95%.

### Example 4

### Astringency Properties

Quartz Crystal Microbalance with Dissipation monitoring (QCM-D) is a real-time, nanoscale technique for monitoring and characterizing thin films on surfaces as well for analyzing surface phenomena including thin film formation, adsorption, desorption, molecular interactions, reactions and structural properties. In this way, the QCM operates as a very sensitive balance.

A QCM sensor consists of a thin quartz disc sandwiched between a pair of electrodes. Due to the piezoelectric properties of quartz, it is possible to excite the crystal to oscillation by applying an AC voltage across its electrodes. Normally the electrodes are made of gold, which can be coated with a wide range of different materials. The resonance frequency (f) of the sensor depends on the total oscillating mass, including water coupled to the oscillation. When a thin film is attached to the sensor, the frequency decreases. If the film is thin and rigid the decrease in frequency is proportional to the mass of the film. Frequency monitoring gives information about mass changes and thickness of adsorbed film. Adsorbed films dampen the sensor's oscillation. The damping or energy dissipation (D) of the sensor's oscillation reveals the film's softness (viscoelasticity). Therefore dissipation is related to the rigidity of the adsorbed film.

This technique was used to analyse the properties of adsorbed BWAT Raspberry water films on a β-Lactoglobulin layer in order to compare the obtained physico-chemical data to the sensory data mentioned beforehand. β-Lactoglobulin was used in order to mimic the protein layer in the oral cavity.

The measurements were done as duplicates. First a protein layer was adsorbed on the quartz crystal. As soon as equilibrium is obtained, the excessive protein layer was rinsed with Millipore water in order to create a homogenous protein monolayer. Ensuing, the Raspberry water was left to rinse over the protein layer—frequency changes and energy dissipation were monitored during a time lapse of 10 min. A last rinsing step with Millipore was conducted.

To understand the role of glucose-6-O-octanoate (G60) as astringency/bitterness modulator the following solutions were investigated on their adsorption behavior. Table 15 shows the mass absorption of a protein layer for a BWAT test solution (without G60) and two BWAT solutions with 200 ppm and 1000 ppm G60, respectively.

TABLE 15

| | mass/area |
|---|---|
| | (ng/cm²) |
| BWAT Test (no G60) | 1082 |
| BWAT w/ 200 ppm G60 | 1257 |
| BWAT w/ 1000 ppm G60 | 1194 |

A higher mass adsorption compared to the control sample is observed in presence of G60. Over the 10min of mass adsorption monitoring the adsorbed mass starts to get rinsed off again. The higher the G60 concentration the lower is the amount of rinsed away mass.

Correlating these date with the sensory evaluation it can be assumed that the permanently bound layer in presence of G60 influences the perception of astringency and bitterness: the thicker the adsorbed film and the higher the adsorbed mass on the protein layer the lower is the sensory impact of the astringent flavor component.

The correlation between sensory data and QCM-D is herewith described for the first time. The QCM-D allows therefore a physico-chemical approach of astringency evaluation.

### Additional Embodiments

G1. A flavor-enhancing compound, wherein the flavor-enhancing compound is a monoester derivative of a primary alcohol residue of a sugar compound having a sugar moiety and an acid moiety.
G2. The flavor-enhancing compound of G1, wherein the sugar moiety is a monosaccharide moiety or a disaccharide moiety.
G3. The flavor-enhancing compound of G2, wherein the sugar moiety is a monosaccharide moiety.
G4. The flavor-enhancing compound of G3, wherein the sugar moiety is a pentose moiety or a hexose moiety.
G5. The flavor-enhancing compound of G4, wherein the sugar moiety is a pentose moiety.
G6. The flavor-enhancing compound of G5, wherein the sugar moiety is an arabinose moiety, a lyxose moiety, a ribose moiety, a xylose moiety, a ribulose moiety, a xylulose moiety, or a deoxuribose moiety.
G7. The flavor-enhancing compound of G4, wherein the sugar moiety is a hexose moiety.
G8. The flavor-enhancing compound of G7, wherein the sugar moiety is an allose moiety, an altrose moiety, a glucose moiety, a mannose moiety, a gulose moiety, an idose moiety, a galactose moiety, a talose moiety, a psicose moiety, a fructose moiety, a sorbose moiety, or a tagatose moiety.
G9. The flavor-enhancing compound of G8, wherein the sugar moiety is a glucose moiety.
G10. The flavor-enhancing compound of G3, wherein the sugar moiety is a fructose moiety, a sorbose moiety, a tagatose moiety, a psicose moiety, an allose moiety, an altrose moiety, a glucose moiety, a mannose moiety, a gulose moiety, an idose moiety, a galactose moiety, or a talose moiety.
G11. The flavor-enhancing compound of any one of G3 to G10, wherein the sugar moiety is in a D stereochemical configuration.
G12. The flavor-enhancing compound of any one of G3 to G10, wherein the sugar moiety is in a L stereochemical configuration.
G13. The flavor-enhancing compound of any one of G1 to G12, wherein the acid moiety is a C₆₋₂₄ fatty acid moiety.
G14. The flavor-enhancing compound of G13, wherein the acid moiety is a hexanoate moiety, an octanoate moiety, a decanoate moiety, a 9-decenoate moiety, a 10-undecenoate moiety, a dodecanoate moiety, a 9-dodecenoate moiety, a tetradecanoate moiety, a hexadecanoate moiety, an octadecanoate moiety, an oleate moiety, a linoleate moiety, a linolenate moiety, an eicosapentaenoate moiet, or a docosahexaenoate moiety.
G15. The flavor-enhancing compound of any one of G1 to G12, wherein the acid moiety is an acid moiety of a natural oil.
G16. The flavor-enhancing compound of G15, wherein the natural oil is a vegetable oil, an algae oil, a fish oil, a tall oil, or an animal fat.
G17. The flavor-enhancing compound of G16, wherein the natural oil is rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, or castor oil.
G18. The flavor-enhancing compound of any one of G1 to G12, wherein the acid moiety is an acetate moiety, a hexanoate moiety, an octanoate moiety, a decanoate moiety, an oleate moiety, a palmitate moiety, a hexadecanoate moiety, a laurate moiety, a myristate moiety, a fatty acid moiety of butter oil, a fatty acid moiety of olive oil, a phenylpropanoate moiety, a cinnamate moiety, a caffeate moiety, a gallate moiety, or a ferulate moiety.
G19. The flavor-enhancing compound of G1, wherein the compound is selected from the group consisting of: D-glucose-6-O-acetate, D-glucose-6-O-hexanoate, D-glucose-6-O-octanoate, D-glucose-6-O-decanoate, D-glucose-6-O-laurate, D-glucose-6-O-myristate, D-glucose-6-O-palmitate, D-glucose-6-O-oleate, D-glucose-6-O-3-phenylpropanoate, and D-glucose-6-O-cinnamate.
G20. The flavor-enhancing compound of G1, wherein the compound is D-glucose-6-O-octanoate.
G21. A flavored article comprising one or more flavor-enhancing compounds of any one of G1 to G20.
G22. The flavored article of G21, wherein the flavor-enhancing compounds are present in the flavored article at a concentration of no more than 950 ppm, based on the total weight of the flavored article.
G23. The flavored article of G22, wherein the flavor-enhancing compounds are present in the flavored article at a concentration ranging from 50 ppm to 500 ppm, based on the total weight of the flavored article.
G24. A method for improving a flavor profile of a flavored article, the method comprising:

providing a flavored article, and introducing one or more flavor-enhancing compounds of any one of G1 to G20 to the flavored article.
G25. The method of G24, wherein the introducing comprises introducing the flavor-enhancing compounds to the flavored article at a concentration of no more than 950 ppm, based on the total weight of the flavored article.
G26. The method of G24, wherein the introducing comprises introducing the flavor-enhancing compounds to the flavored article at a concentration ranging from 50 ppm to 500 ppm, based on the total weight of the flavored article.
G27. A method for improving a flavor profile of a flavored article, the method comprising: providing a mixture comprising one or more ingredients for making a flavored article, and introducing one or more flavor-enhancing compounds of any one of G1 to G20 to the mixture to form a flavor-enhanced mixture; and using the flavor-enhanced mixture to form a flavored article.
G28. The method of G27, wherein the introducing comprises introducing the flavor-enhancing compounds to the mixture at a concentration of no more than 950 ppm, based on the total weight of the flavor-enhanced mixture.
G29. The method of G27, wherein the introducing comprises introducing the flavor-enhancing compounds to the mixture at a concentration ranging from 50 ppm to 500 ppm, based on the total weight of the flavor-enhanced mixture.
G30. A method for improving a flavor perception of a flavored article in a human subject, the method comprising: administering a flavored article to a human subject, wherein the flavored article comprises one or more flavor-enhancing compounds of any one of G1 to G20.
G31. The method of G30, wherein the flavor-enhancing compounds are present in the flavored article at a concentration of no more than 950 ppm, based on the total weight of the flavored article.
G32. The method of G30, wherein the flavor-enhancing compounds are present in the flavored article at a concentration ranging from 50 ppm to 500 ppm, based on the total weight of the flavored article.
G33. A method for reducing or masking an off-note of an artificial sweetener, the method comprising: providing a composition comprising an artificial sweetener, and introducing one or more flavor-enhancing compounds of any one of G1 to G20 to the composition.
G34. The method of G33, wherein the introducing comprises introducing the flavor-enhancing compounds to the composition at a concentration of no more than 950 ppm, based on the total weight of the composition.
G35. The method of G33, wherein the introducing comprises introducing the flavor-enhancing compounds to the composition at a concentration ranging from 50 ppm to 500 ppm, based on the total weight of the composition.
G36. The method of any one of G33 to G35, wherein the artificial sweetener is sucralose, acesulfame K, aspartame, a steviol glycoside (such as rebaudioside A, rebaudiosode D, or rebaudioside M), a mogroside, or any combination thereof.
G37. A method for reducing or masking an off-note perception of an artificial sweetener in a human subject, the method comprising: administering a composition to a subject, wherein the composition comprises one or more flavor-enhancing compounds of any one of G1 to G20 and an artificial sweetener.
G38. The method of G37, wherein the flavor-enhancing compounds are present in the composition at a concentration of no more than 950 ppm, based on the total weight of the composition.
G39. The method of G37, wherein the flavor-enhancing compounds are present in the composition at a concentration ranging from 50 ppm to 500 ppm, based on the total weight of the composition.
G40. The method of any one of G37 to G39, wherein the artificial sweetener is sucralose, acesulfame K, aspartame, a steviol glycoside (such as rebaudioside A, rebaudiosode D, or rebaudioside M), a mogroside, or any combination thereof.

1. A flavored article comprising a flavor-enhancing compound, wherein the flavor-enhancing compound is a monoester derivative of a primary alcohol residue of a sugar compound, which comprises a sugar moiety and an acid moiety.

2. The flavored article of claim 1, wherein the sugar moiety of the flavor-enhancing compound is a monosaccharide moiety.

3. The flavored article of claim 2, wherein the sugar moiety of the flavor-enhancing compound is a hexose moiety.

4. The flavored article of claim 3, wherein the sugar moiety of the flavor-enhancing compound is an allose moiety, an altrose moiety, a glucose moiety, a mannose moiety, a gulose moiety, an idose moiety, a galactose moiety, a talose moiety, a psicose moiety, a fructose moiety, a sorbose moiety, or a tagatose moiety.

5. The flavored article of claim 4, wherein the sugar moiety of the flavor-enhancing compound is a glucose moiety.

6. The flavored article of any one of claims 1 to 5, wherein the acid moiety of the flavor-enhancing compound is a C₆₋₂₄ fatty acid moiety.

7. The flavored article of claim 6, wherein the acid moiety of the flavor-enhancing compound is a hexanoate moiety, an octanoate moiety, a decanoate moiety, a 9-decenoate moiety, a 10-undecenoate moiety, a dodecanoate moiety, a 9-dodecenoate moiety, a tetradecanoate moiety, a hexadecanoate moiety, an octadecanoate moiety, an oleate moiety, a linoleate moiety, a linolenate moiety, an eicosapentaenoate moiet, or a docosahexaenoate moiety.

8. The flavored article of any one of claims 1 to 5, wherein the acid moiety of the flavor-enhancing compound is an acetate moiety, a hexanoate moiety, an octanoate moiety, a decanoate moiety, an oleate moiety, a palmitate moiety, a hexadecanoate moiety, a laurate moiety, a myristate moiety, a fatty acid moiety of butter oil, a fatty acid moiety of olive oil, a phenylpropanoate moiety, a cinnamate moiety, a caffeate moiety, a gallate moiety, or a ferulate moiety.

9. The flavored article of claim 1, wherein the flavor-enhancing compound is selected from the group consisting of: D-glucose-6-O-acetate, D-glucose-6-O-hexanoate, D-glucose-6-O-octanoate, D-glucose-6-O-decanoate, D-glucose-6-O-laurate, D-glucose-6-O-myristate, D-glucose-6-O-palmitate, D-glucose-6-O-oleate, D-glucose-6-O-3-phenylpropanoate, and D-glucose-6-O-cinnamate.

10. The flavored article of claim 9, wherein the flavor-enhancing compound is D-glucose-6-O-octanoate.

11. The flavored article of any one of claims 1 to 10, wherein the flavor-enhancing compound is present in the flavored article at a concentration of no more than 950 ppm, based on the total weight of the flavored article.

12. The flavored article of claim 11, wherein the flavor-enhancing compound is present in the flavored article at a concentration ranging from 50 ppm to 500 ppm, based on the total weight of the flavored article.

13. The flavored article of any one of claims 1 to 12, further comprising a natural sweetener, an artificial sweetener, or any combination thereof.

14. The flavored article of any one of claims 1 to 12, further comprising an artificial sweetener.

15. The flavored article of claim 14, wherein the artificial sweetener is selected from the group consisting of: sucralose, aspartame, acesulfame K, steviol glycosides, mogrosides, and any combination thereof.
